SEQUENCE LISTING (14 sequences)

SEQ ID NO: 1 - DNA, 1187 bp, Artificial Sequence
  CMV Promoter With the Beta-Globin Intron Nucleic Acid Sequence from pCMV-MCS (Stratagene)
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccaaacttaagctt

SEQ ID NO: 2 - protein, 451 aa, Artificial Sequence
  Anti-HER2 Antibody Heavy Chain Protein Sequence Without a Signal Peptide, derived from DrugBank
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 16 aa, Artificial Sequence
  Anti-HER2 Antibody Heavy Chain Signal Peptide Protein Sequence
MGWSCIILFLVATATG

SEQ ID NO: 4 - protein, 467 aa, Artificial Sequence
  Anti-HER2 Antibody Heavy Chain Protein Sequence With a Signal Peptide
MGWSCIILFLVATATGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, Artificial Sequence
  Anti-HER2 Antibody Light Chain Protein Sequence Without a Signal Peptide, derived from DrugBank
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 16 aa, Artificial Sequence
  Anti-HER2 Antibody Light Chain Signal Peptide Protein Sequence
MGWSCIILFLVATATG

SEQ ID NO: 7 - protein, 230 aa, Artificial Sequence
  Anti-HER2 Antibody Light Chain Protein Sequence With a Signal Peptide
MGWSCIILFLVATATGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 1353 bp, Artificial Sequence
  Anti-HER2 Antibody Heavy Chain Nucleic Acid Sequence Without a Signal Peptide, derived from DrugBank
gaggtgcagctggtggagtccggcggcggcctggtgcagcccggcggctccctgcggctgtcctgcgccgcctccggcttcaacatcaaggacacctacatccactgggtgcggcaggcccccggcaagggcctggagtgggtggcccggatctaccccaccaacggctacacccggtacgccgactccgtgaagggccggttcaccatctccgccgacacctccaagaacaccgcctacctgcagatgaactccctgcgggccgaggacaccgccgtgtactactgctcccggtggggcggcgacggcttctacgccatggactactggggccagggcaccctggtgaccgtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 9 - DNA, 48 bp, Artificial Sequence
  Anti-HER2 Antibody Heavy Chain Signal Peptide Nucleic Acid Sequence
atgggctggtcctgcatcatcctgttcctggtggccaccgccaccggc

SEQ ID NO: 10 - DNA, 1398 bp, Artificial Sequence
  Anti-HER2 Antibody Heavy Chain Nucleic Acid Sequence With a Signal Peptide
atgggctggtcctgcatcatcctgttcctggtggccaccgccaccggcgaggtgcagctggtggagtccggcggcggcctggtgcagcccggcggctccctgcggctgtcctgcgccgcctccggcttcaacatcaaggacacctacatccactgggtgcggcaggcccccggcaagggcctggagtgggtggcccggatctaccccaccaacggctacacccggtacgccgactccgtgaagggccggttcaccatctccgccgacacctccaagaacaccgcctacctgcagatgaactccctgcgggccgaggacaccgccgtgtactactgctcccggtggggcggcgacggcttctacgccatggactactggggccagggcaccctggtgaccgtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 11 - DNA, 642 bp, Artificial Sequence
  Anti-HER2 Antibody Light Chain Nucleic Acid Sequence Without a Signal Peptide, derived from DrugBank
gacatccagatgacccagtccccctcctccctgtccgcctccgtgggcgaccgggtgaccatcacctgccgggcctcccaggacgtgaacaccgccgtggcctggtaccagcagaagcccggcaaggcccccaagctgctgatctactccgcctccttcctgtactccggcgtgccctcccggttctccggctcccggtccggcaccgacttcaccctgaccatctcctccctgcagcccgaggacttcgccacctactactgccagcagcactacaccaccccccccaccttcggccagggcaccaaggtggagatcaagcggaccgtggccgccccctccgtgttcatcttccccccctccgacgagcagctgaagtccggcaccgcctccgtggtgtgcctgctgaacaacttctacccccgggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccggggcgagtgc

SEQ ID NO: 12 - DNA, 48 bp, Artificial Sequence; identical to SEQ ID NO: 9
  Anti-HER2 Antibody Light Chain Signal Peptide Nucleic Acid Sequence

SEQ ID NO: 13 - DNA, 690 bp, Artificial Sequence
  Anti-HER2 Antibody Light Chain Nucleic Acid Sequence With a Signal Peptide
atgggctggtcctgcatcatcctgttcctggtggccaccgccaccggcgacatccagatgacccagtccccctcctccctgtccgcctccgtgggcgaccgggtgaccatcacctgccgggcctcccaggacgtgaacaccgccgtggcctggtaccagcagaagcccggcaaggcccccaagctgctgatctactccgcctccttcctgtactccggcgtgccctcccggttctccggctcccggtccggcaccgacttcaccctgaccatctcctccctgcagcccgaggacttcgccacctactactgccagcagcactacaccaccccccccaccttcggccagggcaccaaggtggagatcaagcggaccgtggccgccccctccgtgttcatcttccccccctccgacgagcagctgaagtccggcaccgcctccgtggtgtgcctgctgaacaacttctacccccgggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccggggcgagtgc

SEQ ID NO: 14 - DNA, 298 bp, Artificial Sequence
  Bovine Poly(A) Tail Nucleic Acid Sequence
gctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaanccagctggggctcgagggccatacaggccggtacc

(19) United States
(12) Patent Application Publication
(10) Pub. No.: US 2014/0086840 A1
(43) Pub. Date: Mar. 27, 2014

(54) Therapeutic Antibody Target Validation and Screening In Vivo

(71) Applicant: FIVE PRIME THERAPEUTICS, INC., South San Francisco, CA (US)

(72) Inventors: Lewis T. Williams, Mill Valley, CA (US); Justin Wong, Oakland, CA (US); Hongbing Zhang, Albany, CA (US)

(73) Assignee: FIVE PRIME THERAPEUTICS, INC., South San Francisco, CA (US)

(21) Appl. No.: 14/019,684

(22) Filed: Sep. 6, 2013

Related U.S. Application Data
(62) Division of application No. 13/378,318, filed on Feb. 28, 2012, now abandoned.
(60) Provisional application No. 61/220,954, filed on Jun. 26, 2009.

(51) Int. Cl. A61K 49/00

(52) U.S. Cl. CPC A61K 49/0008
USPC 424/9.2

(57) ABSTRACT

An in vivo method of validating a candidate therapeutic target molecule is provided. An in vivo method of selecting a therapeutic antibody to a specific target molecule is also provided.

This application is a divisional of U.S. patent application Ser. No. 13/378,318, filed Feb. 28, 2012, which is a §371 U.S. National Entry of International Patent Application No. PCT/US2010/040029, filed Jun. 25, 2010, which claims priority to U.S. Provisional Application No. 61/220,954, filed Jun. 26, 2009, each of which are incorporated herein by reference.

### TECHNICAL FIELD

An in vivo method of validating a candidate therapeutic target molecule is provided. An in vivo method of screening for a therapeutic antibody to a target molecule is also provided.

### BACKGROUND

Developing a novel antibody therapeutic is typically a very time-consuming and expensive process. In order to select an appropriate target molecule for the antibody therapeutic, the target molecule must be validated, i.e., it must be determined that targeting the molecule will result in alleviation of, or a decrease in the progression of, a selected disease. What constitutes target validation can vary enormously, from experiments to show that the target has a particular function, suggesting that an antibody to the target may be useful for increasing or decreasing that function; to in vitro experiments showing that antibodies to the target have the desired effect on target function; to in vivo animal experiments using injected purified antibodies; to phase II and III clinical trials in humans.

As the validation experiments get closer to mimicking treatment of the disease with an antibody therapeutic in humans, it becomes more and more likely that an antibody therapeutic will be effective. Thus, target validation using in vivo animal experiments are preferable to, for example, target validation using in vitro cell lines. Traditional in vivo target validation is much more expensive, however, in part because larger quantities of purified antibodies must be produced. Furthermore, sufficient quantities of antibodies often cannot be produced by transient expression in cell lines, so stable cell lines must first be established in order to produce antibodies for in vivo experiments. Accordingly, significant time and expense must be incurred before it has even been determined that the selected target molecule is a suitable target for an antibody therapeutic. If the target molecule is found not to be suitable, the entire process must be started again.

In addition, once a target molecule has been validated, antibodies to that target molecule must be screened in order to select a lead candidate for the antibody therapeutic. Researchers typically use in vitro experiments in order to screen antibodies, because it is too expensive and time-consuming to produce and purify dozens of antibodies for testing in vivo. Thus, the lead candidate for the antibody therapeutic is selected based on in vitro experiments, which may not adequately predict the antibody's in vivo efficacy. If the lead candidate is later found not to be suitable in the in vivo model, another antibody from the screen must be selected, produced, purified, and tested in vivo. In some instances, the entire screen must be repeated using different criteria for selecting the lead candidate because the criteria used in the initial screen did not correlate well with in vivo efficacy.

Thus, despite the wide-spread use of in vitro methods for antibody target validation and antibody screening prior to testing in vivo, there remains a need in the art for efficient, low cost, high throughput in vivo methods that can be used to validate a candidate therapeutic antibody target and in screening for a lead therapeutic antibody to a specific target without the need for producing and purifying significant quantities of antibodies prior to validation or screening.

### SUMMARY

The present invention provides a method of validating a candidate target molecule for an antibody therapeutic in vivo without the need for producing and purifying significant quantities of antibodies prior to validation. A candidate target molecule is a therapeutic target for which there is no published report demonstrating in vivo efficacy in the treatment of a disease using an antibody that binds to the candidate target molecule. A candidate target molecule can be validated if an antibody that binds to the candidate target molecule is shown to exhibit in vivo efficacy in the treatment of the disease. The method of the present invention comprises injecting a selected animal model with one or more nucleic acids encoding an antibody that binds to the candidate target molecule. The method further comprises determining whether the encoded antibody is efficacious in the selected animal model, thus validating the candidate target molecule before the time and expense are invested to produce and purify antibodies in large quantities.

In certain embodiments, the present invention also provides a method of screening antibodies to a selected candidate target molecule for efficaciousness in vivo without the need for producing and purifying significant quantities of antibodies prior to validation. Multiple antibodies may be screened in parallel and compared in order to select an antibody that exhibits an appropriate balance between high efficaciousness and low side effects (e.g., toxicity), often called a “lead antibody.” For each antibody, a selected animal model is injected with one or more nucleic acids encoding the antibody and the efficacy of the antibody in the selected animal model is determined. The results of the in vivo efficacy assay for all of the antibodies are then compared to select the antibody having the most desirable properties. In this way, an antibody having particularly desirable properties in vivo can be selected without having to invest the time and expense into producing and purifying many different antibodies in significant quantities.

In certain embodiments, the present invention provides methods of screening antibodies to identify one or more antibodies that exhibit lower toxicity and/or lower side effects. In certain embodiments, the present invention provides screening methods for the early detection of toxicity and/or side effects associated with a candidate antibody. In certain embodiments, a lead antibody is not the most efficacious, but it exhibits lower toxicity and/or lower side effects than a more efficacious antibody. In certain such embodiments, an antibody with lower efficacy may be selected because it exhibits other desirable properties, such as lower toxicity and/or lower side effects.

In certain embodiments, the target molecule has already been validated before the antibodies are screened by a method of the present invention. In certain embodiments, the target molecule has not already been validated, so validation of the target molecule and screening of the antibodies to select an antibody that exhibits an appropriate balance between efficaciousness and low side effects in vivo occur in the same experiment.

As described herein, the inventors have found that injection of a DNA vector encoding an anti-HER2 therapeutic antibody leads to high, sustained expression of full-length anti-HER2 antibody in the plasma of recipient mice. The inventors additionally found that the anti-HER2 antibody expressed in the mouse plasma is properly assembled, comprising two heavy chains and two light chains, as would be expected for a full-length antibody. Further, the inventors have shown that the anti-HER2 antibody in plasma samples is capable of binding to its antigen, HER2, on the surface of HT-B-30 cells. Finally, the inventors have shown that injection of the DNA vector encoding anti-HER2 antibody inhibits primary tumor growth in the JIMT-1 mouse xenograft model of human breast cancer. Thus, hydrodynamic delivery of a nucleic acid encoding the anti-HER2 antibody led to the expression of anti-HER2 antibody, which assembled properly, bound to the HER2 antigen on the surface of HT-B-30 cells, and inhibited primary tumor growth in a mouse xenograft model of human breast cancer.

Taken together, the results of these experiments demonstrate that injection of a nucleic acid encoding a therapeutic antibody can lead to high, sustained expression of a functional full-length antibody, which assembles properly, binds antigen, and exhibits therapeutic activity. Thus, this alternative approach provides an efficient, cost-effective, high throughput in vivo method to validate a candidate therapeutic antibody target and to screen for antibodies to a known therapeutic target using animal models of human disease without the need for producing and purifying significant quantities of antibodies prior to validation or screening.

The present invention includes methods of validating a candidate tumor therapy target molecule in vivo. In certain embodiments, a method comprises injecting a composition into a mouse comprising a human tumor xenograft, wherein the composition comprises a first nucleic acid that encodes an antibody heavy chain and a second nucleic acid that encodes an antibody light chain, and wherein the antibody binds to the candidate tumor therapy target molecule. In certain embodiments, the method further comprises measuring the size of the human tumor xenograft after a period of time. In certain embodiments, the method further comprises comparing the size of the human tumor xenograft to the size of a control human tumor xenograft in a control mouse. In certain embodiments, the method comprises validating the candidate tumor therapy target molecule if the size of the human tumor xenograft is smaller than the size of the control human tumor xenograft. In certain such embodiments, the first nucleic acid encodes an antibody heavy chain variable region, and the second nucleic acid encodes an antibody light chain variable region. In certain embodiments, the first nucleic acid encodes an antibody heavy chain variable region and an antibody heavy chain constant region, and the second nucleic acid encodes an antibody light chain variable region and an antibody light chain constant region. In certain embodiments, the injecting comprises hydrodynamic transfection. In certain embodiments, the injecting comprises hydrodynamic tail vein transfection. In certain embodiments, the first nucleic acid is a first minicircle DNA vector and the second nucleic acid is a second minicircle DNA vector.

In certain embodiments, a method comprises generating a control mouse by injecting a control composition into a mouse comprising a control human tumor xenograft, wherein the control composition comprises a third nucleic acid comprising a nucleic acid that encodes a control antibody heavy chain and a fourth nucleic acid comprising a nucleic acid that encodes a control antibody light chain, wherein the control antibody does not bind to the candidate tumor therapy target molecule. In certain embodiments, the third nucleic acid encodes a control antibody heavy chain variable region, and the fourth nucleic acid encodes a control antibody light chain variable region. In certain embodiments, the third nucleic acid encodes a control antibody heavy chain variable region and a control antibody heavy chain constant region, and the fourth nucleic acid encodes a control antibody light chain variable region and a control antibody light chain constant region. In certain embodiments, the injecting a control composition comprises hydrodynamic transfection. In certain embodiments, the injecting a control composition comprises hydrodynamic tail vein transfection. In certain embodiments, the third nucleic acid is a third minicircle DNA vector and the fourth nucleic acid is a fourth minicircle DNA vector. In certain embodiments, the human tumor xenograft and the control human tumor xenograft are the same type of human tumor xenograft.

In certain embodiments, the present invention provides a method of validating a candidate tumor therapy target molecule in vivo, comprising injecting a composition into a mouse comprising a human tumor xenograft, wherein the composition comprises a nucleic acid that encodes an antibody heavy chain and an antibody light chain, wherein the antibody binds to the candidate tumor therapy target molecule. In certain embodiments, the method comprises measuring the size of the human tumor xenograft after a period of time. In certain embodiments, the method comprises comparing the size of the human tumor xenograft to the size of a control human tumor xenograft in a control mouse. In certain embodiments, the method comprises validating the candidate tumor therapy target molecule if the size of the human tumor xenograft is smaller than the size of the control human tumor xenograft. In certain embodiments, the nucleic acid encodes an antibody heavy chain variable region and an antibody light chain variable region. In certain embodiments, the nucleic acid encodes an antibody heavy chain variable region, an antibody light chain variable region, and a flexible linker that connects the antibody heavy chain variable region and the antibody light chain variable region. In certain embodiments, the nucleic acid encodes an antibody heavy chain variable region and an antibody heavy chain constant region; and an antibody light chain variable region and an antibody light chain constant region. In certain embodiments, the injecting comprises hydrodynamic transfection. In certain embodiments, the injecting comprises hydrodynamic tail vein transfection. In certain such embodiments, the nucleic acid is a minicircle DNA vector.

In certain embodiments, a method comprises generating a control mouse by a method comprising injecting a control composition into a mouse comprising a control human tumor xenograft, wherein the control composition comprises a control nucleic acid that encodes a control antibody heavy chain and a control antibody light chain, wherein the antibody does not bind to the candidate tumor therapy target molecule. In certain embodiments, the control nucleic acid encodes a control antibody heavy chain variable region and a control antibody light chain variable region. In certain embodiments, the control nucleic acid encodes a control antibody heavy chain variable region, a control antibody light chain variable region, and a flexible linker that connects the control antibody heavy chain variable region and the control antibody light chain variable region. In certain embodiments, the control nucleic acid encodes a control antibody heavy chain variable region and a control antibody heavy chain constant region; and a control antibody light chain variable region and a control antibody light chain constant region. In certain embodiments, the injecting a control composition comprises hydrodynamic transfection. In certain embodiments, the injecting a control composition comprises hydrodynamic tail vein transfection. In certain embodiments, the control nucleic acid is a minicircle DNA vector. In certain embodiments, the human tumor xenograft and the control human tumor xenograft are the same type of human tumor xenograft.

In certain embodiments, the present invention provides a method of screening a plurality of therapeutic antibodies to a target molecule in vivo. In certain embodiment, the method comprises obtaining a plurality of compositions, wherein each composition comprises a heavy chain nucleic acid that encodes an antibody heavy chain and a light chain nucleic acid that encodes an antibody light chain, wherein the antibody binds to the target molecule. In certain embodiments, the method comprises injecting a first composition into a first mouse comprising a first human tumor xenograft, wherein the first composition comprises a first heavy chain nucleic acid that encodes a first antibody heavy chain; and a first light chain nucleic acid that encodes a first antibody light chain, and wherein the first antibody binds to the target molecule. In certain embodiments, the method further comprises injecting a second composition into a second mouse comprising a second human tumor xenograft, wherein the second composition comprises a second heavy chain nucleic acid that encodes a second antibody heavy chain, and a second light chain nucleic acid that encodes a second antibody light chain, and wherein the second antibody binds to the target molecule. In certain embodiments, the method comprises measuring the sizes of the first and second human tumor xenografts after a period of time. In certain embodiments, the method further comprises comparing the sizes of the first and second human tumor xenografts; and selecting the antibody that resulted in the smaller human tumor xenograft. In certain embodiments, the first heavy chain nucleic acid encodes a first antibody heavy chain variable region, and the first light chain nucleic acid encodes a first antibody light chain variable region, and the second heavy chain nucleic acid encodes a second antibody heavy chain variable region, and the second light chain nucleic acid encodes a second antibody light chain variable region. In certain embodiments, the first heavy chain nucleic acid encodes a first antibody heavy chain variable region and a first antibody heavy chain constant region, and the first light chain nucleic acid encodes a first antibody light chain variable region and a first antibody light chain constant region, and the second heavy chain nucleic acid encodes a second antibody heavy chain variable region and a second antibody heavy chain constant region, and the second light chain nucleic acid encodes a second antibody light chain variable region and a second antibody light chain constant region. In certain embodiments, the injecting comprises hydrodynamic transfection. In certain embodiments, the injecting comprises hydrodynamic tail vein transfection. In certain embodiments, the first heavy chain nucleic acid is a first minicircle DNA vector, and the first light chain nucleic acid is a second minicircle DNA vector, and the second heavy chain nucleic acid is a third minicircle DNA vector, and the second light chain nucleic acid is a fourth minicircle DNA vector.

In certain embodiments, the present invention provides a method of screening a plurality of therapeutic antibodies to a target molecule in vivo comprising obtaining a plurality of compositions, wherein each composition comprises a nucleic acid that encodes an antibody heavy chain and an antibody light chain, wherein the antibody binds to the target molecule. In certain embodiments, the method comprises injecting a first composition into a first mouse comprising a first human tumor xenograft, wherein the first composition comprises a first nucleic acid that encodes a first antibody heavy chain and a first antibody light chain, wherein the first antibody binds to the target molecule. In certain embodiments, the method further comprises injecting a second composition into a second mouse comprising a second human tumor xenograft, wherein the second composition comprises a second nucleic acid that encodes a second antibody heavy chain and a second antibody light chain, wherein the second antibody binds to the target molecule. In certain embodiments, the method comprises measuring the sizes of the first and second human tumor xenografts after a period of time. In certain embodiments, the method comprises comparing the sizes of the first and second human tumor xenografts; and selecting the antibody that resulted in the smaller human tumor xenograft. In certain embodiments, the first nucleic acid encodes a first antibody heavy chain variable region and a first antibody light chain variable region, and the second nucleic acid encodes a second antibody heavy chain variable region and a second antibody light chain variable region. In certain embodiments, the first nucleic acid encodes a first antibody heavy chain variable region, a first antibody light chain variable region, and a first flexible linker that connects the first antibody heavy chain variable region and the first antibody light chain variable region, and the second nucleic acid encodes a second antibody heavy chain variable region, a second antibody light chain variable region, and a second flexible linker that connects the second antibody heavy chain variable region and the second antibody light chain variable region. In certain embodiments, the first nucleic acid encodes a first antibody heavy chain variable region and a first antibody heavy chain constant region; and a first antibody light chain variable region and a first antibody light chain constant region, and the second nucleic acid encodes a second antibody heavy chain variable region and a second antibody heavy chain constant region; and a second antibody light chain variable region and a second antibody light chain constant region. In certain embodiments, the injecting comprises hydrodynamic transfection. In certain embodiments, the injecting comprises hydrodynamic tail vein transfection. In certain embodiments, the first nucleic acid is a first minicircle DNA vector, and the second nucleic acid is a second minicircle DNA vector.

The present invention provides a method of validating a candidate target molecule in vivo. In certain embodiments, the method comprises injecting a composition into a mouse with a disease, wherein the composition comprises a first nucleic acid that encodes an antibody heavy chain and a second nucleic acid that encodes an antibody light chain, wherein the antibody binds to the candidate target molecule. In certain embodiments, the method comprises injecting a composition into a mouse with a disease, wherein the composition comprises a nucleic acid that encodes an antibody heavy chain and an antibody light chain, wherein the antibody binds to the candidate target molecule. In certain embodiments, the method further comprises determining the alleviation, inhibition of progression, or decrease in severity of the disease after a period of time. In certain embodiments, the method further comprises comparing the alleviation, inhibition of progression, or decrease in severity of the disease to a control mouse. In certain embodiments, the method comprises validating the candidate target molecule if the alleviation, inhibition of progression, or decrease in severity of the disease is greater than the alleviation, inhibition of progression, or decrease in severity of the disease in the control mouse.

In certain embodiments, the present invention provides a method of screening a plurality of therapeutic antibodies to a target molecule in vivo. In certain embodiments, the method comprises obtaining a plurality of compositions, wherein each composition comprises a heavy chain nucleic acid that encodes an antibody heavy chain and a light chain nucleic acid that encodes an antibody light chain, wherein the antibody binds to the target molecule. In certain embodiments, the method comprises injecting a first composition into a first mouse with a disease, wherein the first composition comprises a first heavy chain nucleic acid that encodes a first antibody heavy chain and a first light chain nucleic acid that encodes a first antibody light chain, wherein the first antibody binds to the target molecule. In certain embodiments, the method further comprises injecting a second composition into a second mouse with the disease, wherein the second composition comprises a second heavy chain nucleic acid that encodes a second antibody heavy chain and a second light chain nucleic acid that encodes a second antibody light chain, wherein the second antibody binds to the target molecule. In certain embodiments, the method comprises determining the alleviation, inhibition of progression, or decrease in severity of the disease after a period of time. In certain embodiments, the method further comprises comparing the alleviation, inhibition of progression, or decrease in severity of the disease of the first and second mice. In certain embodiments, the method comprises selecting the antibody that results in the greater alleviation, inhibition of progression, or decrease in severity of the disease.

In certain embodiments, the method of screening a plurality of therapeutic antibodies to a target molecule in vivo comprises obtaining a plurality of compositions, wherein each composition comprises a nucleic acid that encodes an antibody heavy chain and an antibody light chain, wherein the antibody binds to the target molecule. In certain embodiments, the method comprises injecting a first composition into a first mouse with a disease, wherein the first composition comprises a first nucleic acid that encodes a first antibody heavy chain and a first antibody light chain, wherein the first antibody binds to the target molecule. In certain embodiments, the method further comprises injecting a second composition into a second mouse with the disease, wherein the second composition comprises a second nucleic acid that encodes a second antibody heavy chain and a second antibody light chain, wherein the second antibody binds to the target molecule. In certain embodiments, the method comprises determining the alleviation, inhibition of progression, or decrease in severity of the disease after a period of time. In certain embodiments, the method comprises comparing the alleviation, inhibition of progression, or decrease in severity of the disease of the first and second mice; and selecting the antibody that results in the greater alleviation, inhibition of progression, or decrease in severity of the disease.

### BRIEF DESCRIPTION OF THE DRAWINGS

FIG. 1 shows a diagram of a minicircle DNA vector for expression of anti-HER2 antibody. The vector includes sequences encoding the anti-HER2 antibody light chain and heavy chain, each preceded by a CMV promoter and followed by a poly(A) tail, as described in Example 1.

FIGS. 2A and 2B show the nucleic acid (SEQ ID NOs: 10 and 13) and protein (SEQ ID NOs: 4 and 7) sequences for the anti-HER2 heavy chain and light chain.

FIG. 3 shows anti-HER2 antibody expression levels in plasma from SCID mice following hydrodynamic tail vein injection with the minicircle DNA vector shown in FIG. 1, as described in Example 2.

FIG. 4 shows immunoblots used to determine anti-HER2 antibody composition in plasma from a SCID mouse hydrodynamically transfected by tail vein injection with the minicircle DNA vector shown in FIG. 1. Plasma samples were treated with dithiothreitol (DTT) for selected time periods, separated by non-reducing polyacrylamide gel electrophoresis, and subjected to immunoblot analysis using anti-human Fc antibodies, as described in Example 2.

FIG. 5 shows the results of fluorescence activated cell sorting (FACS) analysis to determine the binding activity of anti-HER2 antibody in plasma samples from SCID mice hydrodynamically transfected by tail vein injection with the minicircle DNA vector, shown in FIG. 1, to HER2 on the surface of HT-B-30 cells, as described in Example 3.

FIG. 6 shows that hydrodynamic tail vein injection of the minicircle DNA vector shown in FIG. 1 inhibits primary tumor growth in the JIMT-1 xenograft model of breast cancer, as described in Example 4.

### DETAILED DESCRIPTION OF CERTAIN EMBODIMENTS

The section headings used herein are for organizational purposes only and are not to be construed as limiting the subject matter described.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

Certain techniques used in connection with recombinant DNA, oligonucleotide synthesis, tissue culture and transformation (e.g., electroporation, lipofection), enzymatic reactions, and purification techniques are known in the art. Many such techniques and procedures are described, e.g., in Sambrook et al. Molecular Cloning: A Laboratory Manual (2nd ed., Cold Spring Harbor Laboratory Press, Cold Spring Harbor, N.Y. (1989)), among other places. In addition, certain techniques for chemical syntheses, chemical analyses, pharmaceutical preparation, formulation, and delivery, and treatment of patients are also known in the art.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As utilized in accordance with the present disclosure, the following terms, unless otherwise indicated, shall be understood to have the following meanings:

The terms “nucleic acid molecule” and “polynucleotide” may be used interchangeably and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides in a nucleic acid molecule or polynucleotide.

The terms “polypeptide” and “protein” are used interchangeably and refer to a polymer of amino acid residues. Such polymers of amino acid residues may contain natural and/or non-natural amino acid residues, and include, but are not limited to, peptides, oligopeptides, dimers, trimers, and multimers of amino acid residues. The terms also include polymers of amino acids that have modifications such as, for example, glycosylation, sialylation, acetylation, phosphorylation, pegylation, and the like.

The term “native polypeptide” refers to a naturally occurring polypeptide.

The term “signal peptide” refers to a sequence of amino acid residues located at the amino terminus of a polypeptide that facilitates secretion of a polypeptide from a mammalian cell. A signal peptide may or may not be cleaved upon export of the polypeptide from the mammalian cell, forming a mature protein. Signal peptides may be natural or synthetic, and they may be heterologous or homologous to the protein to which they are attached. Certain exemplary signal peptides include, but are not limited to, antibody heavy chain and light chain signal peptides. A “signal sequence” refers to a nucleic acid sequence that encodes a signal peptide.

The term “vector” is used to describe a nucleic acid that may be engineered to contain a cloned nucleic acid or nucleic acids that may be propagated in a host cell. A vector may include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that may be used in colorimetric assays, e.g., β-galactosidase). The term vector includes viral or retroviral vectors such as, for example, adenoviral vectors, adeno-associated virus vectors, and lentiviral vectors. The term “expression vector” refers to a vector that is used to express a polypeptide of interest in a host cell.

A “minicircle DNA vector” refers to a circular DNA vector devoid of bacterial DNA sequence that permits gene expression in mammalian cells. In certain embodiments, a minicircle DNA vector permits high, persistent gene expression in mammalian cells. Certain methods for constructing minicircle DNA vectors are described, e.g., in Chen et al., Mol. Ther. 8:495-500 (2003) and U.S. Pat. Appl. No. 2004/0214329 A1. Minicircle DNA vectors are described in greater detail below. See also PCT Publication No. WO 2006/076288.

The term “antibody” refers to an intact antibody or a fragment of an antibody that can compete with the intact antibody for antigen binding. Antibody fragments include, but are not limited to, Fab, Fab′, F(ab′)₂, Fv, scFv, dsFv, Fd, and diabodies, including bivalent diabodies and bispecific diabodies, and other antibody fragments that retain at least a portion of the variable region of an intact antibody. See, for example, Hudson et al. (2003) Nat. Med. 9:129-134. Antibodies also include, but are not limited to, camelid antibodies and single domain antibodies (e.g., nanobodies). In certain embodiments, antibody fragments are produced by chemical or enzymatic cleavage of intact antibodies. In certain embodiments, recombinant DNA techniques are used to produce antibody fragments. The term antibody includes, but is not limited to, full-length antibodies, antibody fragments, chimeric antibodies, human antibodies, and humanized antibodies.

As used herein, the term “heavy chain” refers to a polypeptide comprising sufficient heavy chain variable region sequence to confer antigen specificity either alone or in combination with a light chain.

As used herein, the term “light chain” refers to a polypeptide comprising sufficient light chain variable region sequence to confer antigen specificity either alone or in combination with a heavy chain.

The term “full-length antibody” refers to an antibody comprising two full-length heavy chains and two full-length light chains. In a full-length antibody, each heavy chain and each light chain includes a constant (Fc) region and a variable (Fv) region.

A “chimeric antibody” refers to an antibody that is comprised of components from at least two different sources. In certain embodiments, a chimeric antibody comprises a portion of an antibody derived from a first species fused to a portion of an antibody derived from a second species. In certain embodiments, a chimeric antibody comprises a part of an antibody derived from a non-human animal fused to a part of an antibody derived from a human. In certain such embodiments, a chimeric antibody comprises all or a part of a variable region of an antibody derived from a non-human animal fused to a constant region of an antibody derived from a human. Alternatively, in certain embodiments, a chimeric antibody comprises all or part of a variable region of an antibody derived from a human fused to a constant region of an antibody derived from a non-human animal. A chimeric antibody may include a humanized portion, such as a humanized variable region.

A “humanized antibody” refers to a non-human antibody that has been modified so that it corresponds more closely (in amino acid sequence) to a human antibody. In certain embodiments, amino acid residues located outside of the antigen binding site of the variable region of the non-human antibody are modified. In certain embodiments, a humanized antibody is constructed by replacing all or a part of a complementarity determining region (CDR) of a human antibody with all or a part of a CDR from another antibody, such as a non-human antibody that exhibits the desired antigen binding specificity. In certain embodiments, a humanized antibody comprises variable regions in which all or substantially all of the CDRs correspond to CDRs of a non-human antibody and all or substantially all of the framework regions (FRs) correspond to FRs of a human antibody. In certain embodiments, a humanized antibody further comprises an Fc derived from a human antibody or non-human animal.

The term “human antibody” refers to an antibody that contains only human antibody sequences. In certain embodiments, a human antibody may contain synthetic sequences not found in native antibodies. The term is not limited by the manner in which the antibodies are made. For example, in various embodiments, a human antibody may be made in a transgenic mouse, by phage display, by human B-lymphocytes, or by recombinant methods.

The terms “antigen,” “target molecule,” and “target” are used interchangeably to refer to molecules that are bound by an antibody. Exemplary antigens or target molecules include, but are not limited to, polypeptides, nucleic acids, and polysaccharides.

The term “therapeutic target” or “therapeutic target molecule” refers to a molecule that is believed to play a role in one or more human diseases such that targeting the molecule with an antibody is expected to result in alleviation, or a decrease in the progression, of the disease in vivo. A target molecule is considered to be a therapeutic target molecule when targeting the molecule with an antibody is expected to result in alleviation, or a decrease in the progression or severity, of a disease in an animal model of the disease. Therapeutic targets include, but are not limited to, validated and candidate therapeutic targets. Therapeutic targets also include, but are not limited to, tumor therapy targets.

The term “validated therapeutic target” or “validated target molecule” refers to a therapeutic target for which there is a published report demonstrating in vivo efficacy in the treatment of a disease using an antibody that binds to the therapeutic target.

The terms “candidate therapeutic target” and “candidate target molecule” are used interchangeably to refer to a therapeutic target for which there is no published report demonstrating in vivo efficacy in the treatment of a disease using an antibody that binds to the therapeutic target. A candidate therapeutic target is considered validated if in vivo efficacy in the treatment of a disease using an antibody that binds to the therapeutic target is demonstrated using the validation methods described herein.

The term “tumor therapy target” or “tumor therapy target molecule” refers to a therapeutic target that is believed to play a role in cancer and/or tumor growth. Tumor therapy targets include validated and candidate tumor therapy targets.

The term “validated tumor therapy target” as used herein refers to a tumor therapy target for which there is a published report demonstrating in vivo efficacy in tumor treatment using an antibody that binds to the tumor therapy target.

The term “candidate tumor therapy target” as used herein refers to a tumor therapy target for which there is no published report demonstrating in vivo efficacy in tumor treatment using an antibody that binds to the tumor therapy target. A candidate tumor therapy target is considered validated if in vivo efficacy in tumor treatment using an antibody that binds to the tumor therapy target is demonstrated using the validation methods described herein.

The term “therapeutic antibody” refers to an antibody that binds to a therapeutic target molecule and is expected to result in alleviation, or a decrease in the progression, of a disease in vivo.

The term “antigen-binding site” refers to a portion of an antibody that is capable of specifically binding an antigen. In certain embodiments, an antigen-binding site is provided by one or more antibody variable regions. An antigen binding site may comprise amino acid sequences that are not contiguous on a linear antibody polypeptide, but are in actuality in physical proximity to one another in the three-dimensional structure of an antibody.

The term “epitope” refers to a region or portion of an antigen capable of binding specifically to an antibody. In certain embodiments, an epitope may include chemically active surface groups of the antigen such as, for example, amino acid residues, sugar side chains, phosphoryl, or sulfonyl groups. In certain embodiments, an epitope may have specific three dimensional structural characteristics (e.g., a “conformational” epitope) and/or specific charge characteristics.

An antibody “specifically binds” an antigen when it preferentially recognizes the antigen in a complex mixture of proteins and/or macromolecules. In certain embodiments, an antibody is capable of binding different antigens so long as the different antigens comprise the same epitope. In certain instances, for example, homologous proteins from different species may comprise the same epitope. Thus, an antibody may specifically bind to multiple homologous proteins from different species. in certain embodiments, an antibody specifically binds to a human protein and its mouse homologue. In various embodiments, an antibody that specifically binds an antigen binds to the antigen with a dissociation constant (K_{D}) of ≦1 μM, ≦100 nM, or ≦10 nM.

The terms “hydrodynamic delivery of DNA” and “hydrodynamic transfection of DNA” are used interchangeably and refer to a method of gene transfer that involves the rapid intravenous injection of a large volume of an aqueous solution of DNA into a mouse, a rat, or a rabbit. In certain embodiments, the DNA is a minicircle DNA vector. In certain embodiments, the DNA is injected into the tail vein. Hydrodynamic transfection of DNA provides a safe and efficient means of inducing high levels of gene expression in an animal. See, e.g., Zhang et al. (1999) Hum. Gene Ther. 10(10):1735-1737; Liu et al. (1999) Gene Therapy 6:1258-1266; Zhang et al. (2000) Gene Therapy 7:1344-1349; and U.S. Patent Application No. 2005/0153451 A1.

A “host cell” refers to a cell that may be or has been a recipient of a vector or isolated nucleic acid. Host cells may be prokaryotic cells or eukaryotic cells. Exemplary eukaryotic cells include mammalian cells, such as primate (including human) or non-primate animal cells; fungal cells; plant cells; and insect cells. Certain exemplary host cells include, but are not limited to, E. coli, COS cells, 293 and Chinese hamster ovary (CHO) cells, and their derivatives, such as 293-6E and DG44 cells, respectively, and myeloma cells.

The term “isolated” as used herein refers to a molecule that has been separated from at least some of the components with which it is typically found in nature. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a nucleic acid is referred to as “isolated” when it is not part of the larger nucleic acid (such as, for example, genomic DNA or mitochondrial DNA, in the case of a DNA nucleic acid) in which it is typically found in nature, or is separated from at least some of the components of the cell in which it was produced, e.g., in the case of an RNA nucleic acid. Thus, a DNA nucleic acid that is contained in a vector inside a host cell may be referred to as “isolated” so long as that nucleic acid is not found in that vector in nature.

The term “disease” refers to a pathological condition of a body part, organ, or system that may result from one or more of various causes such as, for example, a genetic condition, an environmental stimulus, or an infection.

The terms “subject” and “patient” are used interchangeably herein to refer to mammals, including, but not limited to, rodents, simians, humans, felines, canines, equines, bovines, porcines, ovines, caprines, mammalian laboratory animals, mammalian farm animals, mammalian sport animals, and mammalian pets.

The terms “animal” and “non-human animal” are used interchangeably and refer to non-human animals. Such animals include, but are not limited to, mice, rats, and rabbits.

The term “cancer” refers to a proliferative disorder associated with uncontrolled cell proliferation, unrestrained cell growth, and decreased cell death/apoptosis. Cancer includes, but is not limited to, breast cancer, prostate cancer, lung cancer, kidney cancer, thyroid cancer, melanoma, follicular lymphomas, carcinomas with p53 mutations, and hormone-dependent tumors, including, but not limited to, colon cancer, cardiac tumors, pancreatic cancer, retinoblastoma, glioblastoma, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, Kaposi's sarcoma, ovarian cancer, leukemia (including acute leukemias (for example, acute lymphocytic leukemia, acute myelocytic leukemia, including myeloblastic, promyelocytic, myelomonocytic, monocytic, and erythroleukemia)) and chronic leukemias (for example, chronic myelocytic (granulocytic) leukemia and chronic lymphocytic leukemia), myelodysplastic syndrome polycythemia vera, lymphomas (for example, Hodgkin's disease and non-Hodgkin's disease), multiple myeloma, Waldenström's macroglobulinemia, heavy chain diseases, and solid tumors, including, but not limited to, sarcomas and carcinomas such as fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, and meningioma.

The term “tumor” is used herein to refer to a group of cells that exhibits abnormally high levels of proliferation and growth. A tumor may be benign, pre-malignant, or malignant; malignant tumor cells are cancerous. Tumor cells may be solid tumor cells or leukemic tumor cells. The term “tumor growth” is used herein to refer to proliferation or growth by a cell or cells of a tumor that leads to a corresponding increase in the size of the tumor.

“Treatment,” as used herein, means alleviation, or a decrease in the progression, of the disease in vivo. Treatment includes, but is not limited to, inhibiting the disease itself, inhibiting the progression of the disease, arresting the development of the disease, or relieving the disease, for example, by causing regression. Treatment also includes, but is not limited to, restoring or repairing a lost, missing, or defective function; or stimulating an inefficient process.

The terms “inhibition” or “inhibit” refer to a decrease or cessation of any phenotypic characteristic or to the decrease or cessation in the incidence, degree, or likelihood of that characteristic. In the context of an interaction between two molecules (such as, for example, an antibody and an antigen, two proteins, or a protein and DNA), “inhibition” refers to a decrease in the number of complexes comprising the two molecules. That is, inhibition includes, but is not limited to, a direct blocking of the interaction between the two molecules, an indirect sequestering of one of the molecules so the two molecules are not in the same space and therefore cannot interact, or a decrease in the amount of one or both of the molecules such that fewer complexes between the molecules are formed.

A “pharmaceutically acceptable carrier” refers to a non-toxic solid, semisolid, or liquid filler, diluent, encapsulating material, formulation auxiliary, or carrier conventional in the art for use with a therapeutic agent. A therapeutic agent together with a pharmaceutically acceptable carrier comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

### Structure of Native Antibodies and Certain Antibody Fragments

A full-length antibody has a tetrameric structure. The tetramer typically comprises two identical pairs of polypeptide chains, each pair having one light chain (in certain embodiments, about 25 kDa) and one heavy chain (in certain embodiments, about 50-70 kDa). In a full-length antibody, a heavy chain comprises a variable region, V_{H}, and a constant region made up of three portions, C_{H}1, C_{H}2, and C_{H}3. The V_{H} domain is located at the amino-terminus of the heavy chain, and the C_{H}3 domain is located at the carboxy-terminus. In a full-length antibody, a light chain comprises a variable region, V_{L}, and a constant region, C_{L}. The variable region of the light chain is located at the amino-terminus of the light chain. The variable regions of each light/heavy chain pair form the antigen binding site. The constant regions are typically responsible for effector function.

Human light chains are typically classified as kappa and lambda light chains. Human heavy chains are typically classified as mu, delta, gamma, alpha, or epsilon, and define the isotype of the antibody as IgM, IgD, IgG, IgA, or IgE, respectively. The IgG isotype has subclasses, including, but not limited to, IgG1, IgG2, IgG3, and IgG4. The IgM isotype has subclasses, including, but not limited to, IgM1 and IgM2. The IgA isotype has subclasses, including, but not limited to, IgA1 and IgA2. Within human light and heavy chains, the variable and constant regions are typically joined by a “J” region of about 12 or more amino acids, and the heavy chain also includes a “D” region of about 10 more amino acids. See, for example, Fundamental Immunology (1989) Ch. 7 (Paul, W., ed., 2nd ed. Raven Press, N.Y.).

The heavy and light chain variable regions exhibit the same general structure in which four relatively conserved framework regions (FRs) are joined by three hypervariable regions, also called complementarity determining regions (CDRs). From amino-terminus to carboxy-terminus, both light and heavy chain variable regions typically comprise the domains FR1, CDR1, FR2, CDR2, FR3, CDR3, and FR4. The CDRs on the heavy chain may be referred to as H1, H2, and H3, whereas the CDRs on the light chain may be referred to as L1, L2, and L3. The assignment of amino acids to each domain is discussed, e.g., in Kabat et al. (1991) Sequences of Proteins of Immunological Interest (National Institutes of Health, Publication No. 91-3242, vols. 1-3, Bethesda, Md.); Chothia and Lesk. (1987) J. Mol. Biol. 196:901-917; and Chothia et al. (1989) Nature 342:878-883. In the present application, the term “CDR” refers to a CDR from either the light or heavy chain, unless otherwise specified.

A “Fab” fragment includes one full-length light chain and the C_{H}1 and variable region of one heavy chain. A “Fab” fragment includes one light chain and one heavy chain that includes additional constant region relative to the heavy chain of a Fab fragment, extending between the C_{H}1 and C_{H}2 domains. An interchain disulfide bond can be formed between two heavy chains of a Fab′ fragment to form a “F(ab′)₂” molecule.

An “Fv” fragment comprises the variable regions from both the heavy and light chains, but lacks the constant regions.

A “single-chain Fv” (“scFv”) fragment comprises heavy and light chain variable regions connected by a flexible linker to form a single polypeptide chain with an antigen-binding region. Exemplary single chain antibodies are discussed, e.g., in WO 88/01649 and U.S. Pat. Nos. 4,946,778 and 5,260,203.

A “disulfide-stabilized Fv” (“dsFv”) fragment comprises an Fv fragment that is stabilized by an engineered interchain disulfide bond that connects structurally conserved regions of the V_{H} and V_{L} domains. See e.g., Brinkman et al. (1993) Proc. Nat'l Acad. Sci. USA 90:7538-7542; Reiter et al. (1994) Prot. Eng. 7:697-704; Reiter et al. (1994) Biochem. 33:5451-5459; and Reiter and Pastan (1996) Clin. Can. Res. 2:245-252.

A “diabody” is a class of small antibody fragments that comprise a heavy chain variable domain connected to a light chain variable domain by a peptide linker on the same polypeptide chain. The peptide linker joining the heavy chain variable domain to the light chain variable domain is too short to allow pairing between the two domains on the same polypeptide chain, which forces pairing with the complementary domains of a second antibody chain and promotes the assembly of a dimeric molecule with two functional antigen binding sites. Diabodies can be “bivalent diabodies” (i.e., when two of the same antibody fragments form a homodimer to produce a diabody with the same two antigen binding sites), or they can be “bispecific diabodies” (i.e., when two different antibody fragments form a heterodimer to produce a diabody with two different antigen binding sites). See, e.g., Holliger et al. (1993) Proc. Nat'l Acad. Sci. USA 90:6444-6448.

A “camelid antibody” is an antibody derived from a camelid animal that lacks a light chain. A single N-terminal domain of a camelid antibody (called VHH or Nanobody) is capable of antigen binding without requiring domain pairing. Camelid antibodies lack a CH1 domain. See e.g., Harmsen et al. (2007) Appl. Microbiol. Biotechnol. 77:13-22.

A “recombinant immunotoxin” or “immunotoxin” is a chimeric protein comprising a toxin, including, but not limited to, Pseudomonas exotoxin or diptheria toxin, and an antigen-binding domain, such as an Fv or Fab, wherein the chimeric protein is translated from a nucleic acid molecule that encodes the toxin and the antigen-binding domain. In certain embodiments, the toxin is truncated and/or mutated. In certain embodiments, the toxin inhibits growth of and/or kills certain target cells. Exemplary antigen-binding domains of the immunotoxin include, but are not limited to, scFv and dsFv. See e.g., Nicholls et al. (1993) J. Biol. Chem. 268(7):5302-5308; Brinkman et al. (1993) Proc. Nat'l Acad. Sci. USA 90:7538-7542; Nagata et al. (2002) Clin. Can. Res. 8:2345-2355; and Kuan et al. (1996) Proc. Nat'l Acad. Sci. USA 93:974-978.

### Methods of Making Monoclonal Antibodies to a Target Molecule

Monoclonal antibodies to a target molecule can be made using standard methods such as, for example, hybridoma-based methods, genetically altered and transgenic mouse-based methods, recombinant methods, and display methods. Human antibodies can be made using methods such as, for example, transgenic mice comprising human heavy chain and light chain loci, human B-lymphocytes, recombinant methods, and display methods.

In certain embodiments, an antibody that cross-reacts with a human target molecule and its mouse homologue can be identified. By testing antibodies that cross-react with both the human target molecule and its mouse homologue in the validation and/or screening methods described herein, the therapeutic antibody identified in a mouse model would be expected to have similar activity in the corresponding human disease.

In certain embodiments, when a human or humanized antibody is used in the validation and/or screening methods described herein, the human Fc can be replaced with a non-human animal Fc during the validation and/or screening process. After a therapeutic antibody is identified by the methods described herein, the non-human animal Fc can be replaced with a desired human Fc for testing in humans. In certain embodiments, the non-human animal Fc is a mouse Fc.

Certain Hybridoma Methods

In certain embodiments, hybridoma-based methods are used to produce monoclonal antibodies. Certain such methods are known to those skilled in the art. See, e.g., Kohler et al. (1975) Nature 256:495-497; Harlow and Lane (1988) Antibodies: A Laboratory Manual Ch. 6 (Cold Spring Harbor Laboratory, Cold Spring Harbor, N.Y.). In certain embodiments, a suitable animal, such as a mouse, rat, hamster, monkey, or other mammal, is immunized with the antigen (i.e., target molecule) to produce antibody-secreting cells. In certain embodiments, the antibody-secreting cells are B-cells, such as lymphocytes or splenocytes. In certain embodiments, lymphocytes (e.g., human lymphocytes) are immunized in vitro to generate antibody-secreting cells. See, e.g., Borreback et al. (1988) Proc. Nat'l Acad. Sci. USA 85:3995-3999.

In certain embodiments, antibody secreting cells are fused with an “immortalized” cell line, such as a myeloid-type cell line, to produce hybridoma cells. Hybridoma cells that produce the desired antibodies may then be identified, for example, by enzyme-linked immunosorbant assay (ELISA). Such cells can be propagated, e.g., by subcloning and culturing using standard methods or grown in vivo as ascites tumors in a suitable animal host. Monoclonal antibodies may then be isolated from hybridoma culture medium, serum, or ascites fluid using standard separation procedures, such as affinity chromatography. Guidance for the production of hybridomas and the purification of monoclonal antibodies according to certain embodiments is provided, for example, in Harlow and Lane (1988) Antibodies: A Laboratory Manual Ch. 8 (Cold Spring Harbor Laboratory, Cold Spring Harbor, N.Y.).

Certain Genetically Altered and Transgenic Mouse-Based Methods

In certain embodiments, human monoclonal antibodies are raised in transgenic animals (e.g., mice) that are capable of producing human antibodies. Such mice may comprises, for example, human heavy chain and light chain loci that are competent to produce human antibodies in the mouse. Certain exemplary methods and transgenic mice suitable for the production of human monoclonal antibodies are described, e.g., in U.S. Pat. Nos. 6,075,181 A and 6,114,598 A; WO 98/24893 A2; Jakobovits et al. (1993) Nature 362:255-258; Tomizuka et al. (2000) Proc. Nat'l Acad. Sci. USA 97:722-727; Jakobovits (1995) Curr. Opin. Biotechnol. 6:561-566; Lonberg et al. (1995) Int'l Rev. Immunol. 13:65-93; Fishwild et al. (1996) Nat. Biotechnol. 14:845-851; Green (1999) J. Immunol. Methods 231:11-23; and Mendez et al. (1997) Nat. Genet. 15:146-156 (describing the XenoMouse II® line of transgenic mice). After immunization of the transgenic mouse with an antigen, antibody-secreting cells may be isolated and propagated as described above in the section titled “Certain Hybridoma Methods.”

Certain Recombinant Methods

In certain embodiments, monoclonal antibodies may be manipulated by recombinant techniques. In certain such embodiments, nucleic acid(s) encoding the heavy chain and light chain of the monoclonal antibody chains may be isolated and cloned from the cell expressing the antibody. For example, RNA can be prepared from cells expressing the desired antibody, such as mature B-cells or hybridoma cells, using standard methods. The RNA can then be used to make cDNA using standard methods, and the cDNA can be amplified, for example, by PCR, using specific oligonucleotide primers. In certain embodiments, the cDNA is then cloned into a vector suitable for the desired application. For example, if the antibody is not going to be modified prior to use in the described validation and/or screening methods, then the heavy and/or light chain cDNA may be cloned into a vector suitable for creating a minicircle DNA, as described below, or into a vector suitable for expression in an animal following injection such as, for example, an adenoviral vector, an adeno-associated virus vector, or a lentiviral vector.

In certain embodiments, cDNA encoding a heavy chain and/or light chain can be modified in order to modify the expressed heavy and/or light chain. For example, in certain embodiments, the constant region of a mouse heavy or light chain can be replaced with the constant region of a human heavy or light chain. In this manner, in certain embodiments, a chimeric antibody can be produced which possesses human antibody constant regions but retains the binding specificity of a mouse antibody. Alternatively, the constant region of a human heavy or light chain can be replaced with the constant region of a non-human animal heavy or light chain. In this case, a chimeric antibody can be produced which possesses non-human animal antibody constant regions, e.g., for expression in a non-human animal model, but retains the binding specificity of the human antibody.

Certain Display-Based Methods

In certain embodiments, monoclonal antibodies are produced using display-based methods such as, for example, phage display, yeast display, bacterial display, and ribosome display. In certain embodiments, a human monoclonal antibody is produced using a display-based method.

In certain embodiments, a monoclonal antibody is produced using phage display techniques. Certain exemplary antibody phage display methods are known to those skilled in the art and are described, for example, in Hoogenboom, “Overview of Antibody Phage-Display Technology and Its Applications,” from Methods in Molecular Biology: Antibody Phage Display: Methods and Protocols (2002) 178:1-37 (O'Brien and Aitken, eds., Human Press, Totowa, N.J.). As an example of an antibody phage display method, a library of antibodies is displayed on the surface of a filamentous phage, such as the nonlytic filamentous phage fd or M13. In certain embodiments, the displayed antibodies are antibody fragments, such as scFvs, Fabs, Fvs (e.g., dsFvs with an engineered intermolecular disulfide bond to stabilize the V_{H}-V_{L} pair), diabodies, and camelid antibodies. Antibodies with the desired binding specificity can then be selected. Certain exemplary embodiments of antibody phage display methods are described in further detail below.

An antibody phage-display library can be prepared, for example, using certain methods known to those skilled in the art. See, e.g., Hoogenboom, “Overview of Antibody Phage-Display Technology and Its Applications,” from Methods in Molecular Biology: Antibody Phage Display: Methods and Protocols (2002) 178:1-37 (O'Brien and Aitken, eds., Human Press, Totowa, N.J.). In certain embodiments, variable gene repertoires are prepared by PCR amplification of genomic DNA or cDNA derived from the mRNA of antibody-secreting cells. For example, in certain embodiments, cDNA is prepared from mRNA of B-cells. The antibody-secreting cells may be isolated from an unimmunized animal or from an animal that has been immunized with the antigen of interest in order to enrich for antigen-binding sequences. Alternatively, “synthetic” antibody libraries may be constructed using repertoires of gene segments that are rearranged in vitro. For example, in certain embodiments, individual gene segments encoding heavy or light chains (V-D-J or V-J, respectively) are randomly combined using PCR. In certain such embodiments, additional sequence diversity can be introduced into the CDRs, and possibly FRs, e.g., by error prone PCR. In some instances, a combination of the two approaches can be used to create “semi-synthetic” libraries, wherein some of the CDRs are derived from naturally occurring sources (e.g., immunized or unimmunized animals), and other CDRs are derived from synthetic sources (e.g., error-prone PCR).

Certain exemplary universal human antibody phage display libraries are available from commercial sources. Certain exemplary libraries include, but are not limited to, the HuCAL® series of libraries from MorphoSys AG (Martinstreid/Munich, Germany); libraries from Crucell (Leiden, the Netherlands) using MAbstract® technology; the n-CoDeR™ Fab library from BioInvent (Lund, Sweden); and libraries available from Cambridge Antibody Technology (Cambridge, UK).

In certain embodiments, the selection of antibodies having the desired binding specificity from a phage display library is achieved by successive panning steps. Such panning steps typically involve exposing library phage preparations to antigen, washing the phage-antigen complexes, and discarding unbound phage. Bound phage may then be recovered and amplified by infecting E. coli. In certain embodiments, the above process is repeated one or more times. In certain embodiments, library phage preparations are exposed in sequence to a matched set of cells, only one of which expresses the antigen of interest (e.g., a matched set of untransfected parental cells and parental cells transfected with a vector for antigen expression). In this case, phage that express antibodies that bind to the untransfected parental cells can be eliminated prior to selecting for those that express antibodies that bind to the target of interest expressed on transfected parental cells. In certain such embodiments, antibodies that may bind to the target of interest but also exhibit nonspecific binding to other targets on the cell surface may be eliminated.

In certain embodiments, a yeast display system is used to produce monoclonal antibodies. In certain such systems, an antibody is expressed as a fusion protein with all or a portion of the yeast AGA2 protein, which becomes displayed on the surface of the yeast cell wall. Yeast cells expressing antibodies with the desired binding specificity are then identified, for example, by exposing the cells to fluorescently labeled antigen. In certain such embodiments, yeast cells that bind the antigen can then be isolated, e.g., by flow cytometry. See, e.g., Boder et al. (1997) Nat. Biotechnol. 15:553-557; and Feldhaus et al. (2003) Nat. Biotechnol. 21:163-170. In certain embodiments, a bacterial display system is used to select monoclonal antibodies. See, e.g., Skerra et al. (1988) Science 240:1038-1041; and Better et al. (1988) Science 240:1041-1043; Harvey et al. (2004) Proc. Nat'l Acad. Sci. USA 101(25):9193-9198; and Mazor et al. (2007) Nat. Biotechnol. 25(5):563-565. In certain embodiments, a ribosome display system is used to select monoclonal antibodies. See, e.g., Hanes et al. (1997) Proc. Nat'l Acad. Sci. USA 94:4937-4942; Schaffitzel (1999) J. Immunol. Meth. 231:119-135; Hanes et al. (2000) Nat. Biotechnol. 18:1287-1292; Lipovsek et al. (2004) J. Immunol. Meth. 290:51-67.

### Certain Methods to Increase Antibody Affinity for a Target Molecule

In certain embodiments, in vitro methods are used to increase the affinity of an antibody for a selected target molecule. In vivo, native antibodies undergo affinity maturation through somatic hypermutation followed by selection. Certain in vitro methods, referred to herein as affinity maturation (or “directed evolution”), mimic that in vivo process, thereby allowing the production of antibodies having affinities equivalent to or surpassing those of native antibodies. In certain embodiments, libraries of antibodies are created using, for example, phage, ribosome, or yeast display methods, so that antibodies with increased affinity may be identified by standard screening methods.

In certain embodiments of affinity maturation, mutations are introduced into a nucleic acid sequence encoding the variable region of an antibody having the desired binding specificity for a target molecule. See, e.g., Hudson et al. (2003) Nat. Med. 9:129-134; Brekke et al. (2002) Nat. Reviews 2:52-62. Mutations may be introduced into the variable region of the heavy chain, light chain, or both. Further, mutations may be introduced into one or more framework (FR) regions and/or one or more complementarity determining (CDR) regions. In certain embodiments, a library of mutations is created, for example, in a phage, ribosome, or yeast display library, so that antibodies with increased affinity may be identified by standard screening methods. See, e.g., Boder et al. (2000) Proc. Nat'l Acad. Sci. USA 97:10701-10705; Foote et al. (2000) Proc. Nat'l Acad. Sci. USA 97:10679-10681; Hoogenboom, Overview of Antibody Phage-Display Technology and Its Applications, from Methods in Molecular Biology: Antibody Phage Display: Methods and Protocols (2002) 178:1-37 (O'Brien and Aitken, eds., Human Press, Totowa, N.J.); and Hanes et al. (1998) Proc. Nat'l Acad. Sci. USA 95:14130-14135. In certain embodiments, mutations are introduced by amplifying the sequences using error prone polymerases. A nonlimiting example of such a method is described in detail in Hanes et al. (1998) Proc. Nat'l Acad. Sci. USA 95:14130-14135. Alternatively, in certain embodiments, mutations are introduced using E. coli mutator cells or homologous gene rearrangement. In certain embodiments, mutations are introduced using “DNA shuffling.” See, e.g., Crameri et al. (1996) Nat. Med. 2:100-102; Fermer et al. (2004) Tumor Biol. 25:7-13. In certain embodiments, mutations are introduced by site-specific mutagenesis. For example, mutations may be introduced based on information regarding the antibody's structure such as, for example, the antigen binding site.

Certain methods of “chain shuffling” may also be used to generate antibodies with increased affinity. In certain embodiments of chain shuffling, one of the chains, e.g., the light chain, is replaced with a repertoire of light chains, while the other chain, e.g., the heavy chain, is unchanged. In certain such embodiments, a library of chain-shuffled antibodies is created, wherein the unchanged heavy chain is expressed in combination with each light chain from the repertoire of light chains. In certain embodiments, such libraries may then be screened for antibodies with increased affinity. See, e.g., Hudson et al. (2003) Nat. Med. 9:129-134; Brekke et al. (2002) Nat. Reviews 2:52-62; Kang et al. (1991) Proc. Nat'l Acad. Sci. USA 88:11120-11123; Marks et al. (1992) Biotechnol. 10:779-83.

### Chimerized and Humanized Monoclonal Antibodies

In certain embodiments, human or non-human antibodies are chimerized. In certain embodiments, mouse monoclonal antibodies are chimerized by replacing the mouse Fc with a human Fc. In certain embodiments, human monoclonal antibodies are chimerized by replacing the human Fc with a non-human animal Fc. In certain embodiments, the human Fc is replaced with a mouse Fc. Certain exemplary methods for making chimeric antibodies are provided, for example, in Morrison et al. (1984) Proc. Nat'l Acad. Sci. USA 81:6851-6855; Neuberger et al. (1984) Nature 312:604-608; Takeda et al. (1985) Nature 314:452-454; and U.S. Pat. Nos. 6,075,181 and 5,877,397.

In certain embodiments, non-human antibodies are “humanized.” As a non-limiting example, a mouse monoclonal antibody that specifically binds the target molecule may be humanized in order to reduce immunogenicity (e.g., reduced human anti-mouse antibody (HAMA) response) when administered to a human. In certain embodiments, a humanized antibody has a similar binding affinity for the target molecule as the non-humanized parent antibody. In certain embodiments, a humanized antibody has increased binding affinity for the target molecule when compared to the non-humanized parent antibody. Certain exemplary humanization methods include, but are not limited to, CDR grafting and human engineering, as described in detail below.

In certain embodiments, one or more complementarity determining regions (CDRs) from the light chain and/or heavy chain variable regions of an antibody with the desired binding specificity (the “donor” antibody) are grafted onto human framework regions (FRs) of the light and/or heavy chain of an “acceptor” antibody in order to create a humanized antibody with the binding specificity of the donor antibody. Exemplary CDR grafting is described, e.g., in U.S. Pat. Nos. 6,180,370; 5,693,762; 5,693,761; 5,585,089; and 5,530,101; Queen et al. (1989) Proc. Nat'l Acad. Sci. USA 86:10029-10033. In certain embodiments, one or more CDRs from the light and/or heavy chain variable regions of the donor antibody are grafted onto consensus human FRs. To create consensus human FRs, FRs from several human heavy chain or light chain amino acid sequences may be aligned to identify a consensus amino acid sequence.

Additionally, in certain embodiments, certain FR amino acids in the acceptor antibody may be replaced with FR amino acids from the donor antibody, e.g., when those amino acids contribute to the affinity of the donor antibody for the target molecule. See, e.g., in U.S. Pat. Nos. 6,180,370; 5,693,762; 5,693,761; 5,585,089; and 5,530,101; Queen et al. (1989) Proc. Nat'l Acad. Sci. USA 86:10029-10033. In certain embodiments, computer programs are used for modeling donor and/or acceptor antibodies to identify residues that are likely to be involved in binding the target molecule and/or are likely to contribute to the structure of the antigen binding site. In certain embodiments, grafted FRs in a humanized antibody are further modified (e.g., by amino acid substitutions, deletions, or insertions) to increase the affinity of the humanized antibody for the target molecule.

Finally, non-human antibodies may be humanized using a “human engineering” method as described, for example, in U.S. Pat. Nos. 5,766,886 and 5,869,619.

Nucleic Acids that Encode Antibodies

In certain embodiments, the present invention includes a nucleic acid that encodes an antibody heavy chain, an antibody light chain, or both an antibody heavy chain and an antibody light chain. Exemplary nucleic acids include, but are not limited to, plasmid vectors, minicircle DNA vectors, and viral vectors such as, for example, adenoviral vectors, adeno-associated virus vectors, and lentiviral vectors. In certain embodiments, a nucleic acid includes all of the elements required for the proper expression of an antibody heavy chain and/or light chain in a cell or animal. Such elements include, but are not limited to, promoters, enhancers, untranslated regions, ribosome binding sites, etc. A vector may also include a selectable marker for growth in prokaryotic and/or eukaryotic host cells, and/or an origin of replication for propagation in prokarytic and/or eukaryotic host cells.

In certain embodiments, a nucleic acid also encodes at least one selectable marker. Such markers include, but are not limited to, dihydrofolate reductase, G418 or neomycin resistance for eukaryotic cell culture, and tetracycline, kanamycin, or ampicillin resistance genes for culturing in E. coli and other bacteria. In certain embodiments, a vector encodes at least one origin of replication. Such origins of replication allow for the propagation of the vector in a suitable host cell, which can be either a eukaryotic or a prokaryotic cell. Origins of replication are known in the art, as described, for example, in Genes II, Lewin, B., ed., John Wiley & Sons, New York (1985).

In certain embodiments, a nucleic acid encodes a heavy chain only. In certain embodiments, a nucleic acid encodes a light chain only. In certain embodiments, a nucleic acid encodes both an antibody heavy chain and an antibody light chain. When a nucleic acid encodes both an antibody heavy chain and an antibody light chain, in certain embodiments, it may produces two separate transcripts, one for the antibody heavy chain and one for the antibody light chain.

Alternatively, a single transcript may be produced that encodes both the antibody heavy chain and the antibody light chain. When the antibody heavy chain and antibody light chain are encoded by a single transcript, the corresponding polypeptides may be separately expressed, e.g., by including an internal ribosome entry site (IRES) between the antibody heavy chain coding sequence and the antibody light chain coding sequence. In certain embodiments, when the antibody heavy chain and antibody light chain are encoded by a single transcript, the corresponding polypeptides may be expressed as a single polypeptide. In certain such embodiments, the heavy chain and light chain are connected by a flexible linker, such as, for example, an scFv. In certain embodiments, when a single polypeptide is expressed that comprises both the heavy chain and the light chain, a foot-and-mouth-disease virus (FMDV)-derived 2A self-processing sequence located adjacent to a furin cleavage site is located between the antibody heavy chain and light chain, which permits post-translational cleavage, separating the two chains. See, e.g., U.S. Pat. App. Nos. 2004/0265955 A1 and 2005/0003842 A1.

Minicircle Producing Plasmids and Minicircle DNA Vectors

In certain embodiments, a nucleic acid that encodes a heavy chain and/or a light chain is a minicircle DNA vector. A minicircle DNA vector lacks the bacterial sequences that many vectors contain. Minicircle DNA vectors and methods for producing them are described, e.g., in Chen et al., Mol. Ther. 8:495-500 (2003), and U.S. Pat. App. No. 2004/0214329 A1. In certain embodiments, a minicircle DNA vector is less labor-intensive to produce than purified linear vectors. Furthermore, a minicircle DNA vector may be less likely to integrate into the host animal genome than other vectors or linear DNA. See, e.g., Chen et al. (2003) Mol. Ther. 8:495-500).

As a nonlimiting example, a minicircle DNA vector may be produced as follows. An expression cassette, which comprises a coding sequence of interest along with regulatory elements for its expression, is flanked by attachment sites for a recombinase. A sequence encoding the recombinase is located outside of the expression cassette and includes elements for inducible expression (such as, for example, an inducible promoter). Upon induction of recombinase expression, the vector DNA is recombined, resulting in two distinct circular DNA molecules. One of the circular DNA molecules is relatively small, forming a minicircle that comprises the expression cassette for the gene of interest; this minicircle DNA vector is devoid of any bacterial DNA sequences. The second circular DNA sequence contains the remaining vector sequence, including the bacterial sequences and the sequence encoding the recombinase. The minicircle DNA containing the gene of interest can then be separately isolated and purified. In certain embodiments, a minicircle DNA vector may be produced using plasmids similar to pBAD.φC31.hFIX and pBAD.φC31.RHB. See, e.g., Chen et al. (2003) Mol. Ther. 8:495-500.

Exemplary recombinases that may be used for creating a minicircle DNA vector include, but are not limited to, Streptomyces bacteriophage φ31 integrase, Cre recombinase, and the λ integrase/DNA topoisomerase IV complex. Each of these recombinases catalyzes recombination between distinct sites. For example, φ31 integrase catalyzes recombination between corresponding attP and attB sites, Cre recombinase catalyzes recombination between loxP sites, and the λ integrase/DNA topoisomerase IV complex catalyzes recombination between bacteriophage λ attP and attB sites. In certain embodiments, such as, for example, with φ31 integrase or with λ integrase in the absence of the Xis protein, the recombinase mediates an irreversible reaction to yield a unique population of circular products and thus high yields. In other embodiments, such as, for example, with Cre recombinase or with λ integrase in the presence of the Xis protein, the recombinase mediates a reversible reaction to yield a mixture of circular products and thus lower yields. The reversible reaction by Cre recombinase can be manipulated by employing mutant loxP71 and loxP66 sites, which recombine with high efficiency to yield a functionally impaired P71/66 site on the minicircle molecule and a wild-type loxP site on the minicircle molecule, thereby shifting the equilibrium towards the production of the minicircle DNA product.

Certain Methods of Delivering Nucleic Acids into an Animal

Certain exemplary methods of introducing nucleic acids into an animal include, but are not limited to, hydrodynamic tail vein transfection (TVT), viral methods, and standard injection methods. TVT methods may involve the delivery of nucleic acids, including, but not limited to, minicircle DNA vectors, viral vectors, and standard plasmid DNA vectors. Viral methods may involve the delivery of viral vectors, including, but not limited to, adenoviral vectors, adeno-associated virus vectors, and lentiviral vectors. Standard injections may include, but are not limited to, intraperitoneal (i.p.) injections. Standard injections may involve the delivery of nucleic acids, including, but not limited to, minicircle DNA vectors and standard plasmid DNA vectors. The nucleic acids may be introduced one time or multiple times, depending on the length of the experiment. In certain embodiments, a nucleic acid encoding an antibody heavy chain is delivered together with a nucleic acid encoding an antibody light chain. In certain embodiments, a single nucleic acid is delivered which encodes both an antibody heavy chain and an antibody light chain.

Hydrodynamic Delivery of Nucleic Acids by Tail Vein Injection

In certain embodiments, one or more nucleic acids are hydrodynamically delivered by tail vein injection into an animal. In certain embodiments, one or more of the delivered nucleic acids is a minicircle DNA vector. DNA is diluted to 10 ug/ml in saline (0.91% NaCl solution). The DNA is administered as a bolus intravenous injection in the tail vein with approximately 2 ml of the DNA-containing solution (8-10% of volume/body weight). In certain embodiments, one or more of the delivered nucleic acids is an adenoviral vector, an adeno-associated virus vector, or a lentiviral vector.

### Animal Models of Human Disease

In certain embodiments, the present invention uses one or more animal models of disease to validate a candidate target molecule in vivo and/or to screen for therapeutic antibodies in vivo. Such animal models include, but are not limited to, mouse and rat models of disease. Animal models of disease may include any animal model that exhibits a characteristic or characteristics (i.e., phenotype or phenotypes) associated with a human disease or disorder, such as, for example, cancer, arthritis, multiple sclerosis, and diabetes. Many such animal models are known in the art.

In certain embodiments, an animal model is a mouse model of human disease. A variety of mouse models of human disease are known in the art, including, but not limited to, the mouse collagen induced arthritis (CIA) model of rheumatoid arthritis (see, e.g., Hegen et al., (2008) Ann. Rheum. Dis. 67:1505-1515; Williams et al. (1992) Proc. Nat'l Acad. Sci. USA 89(20):9784-9788), the mouse experimental autoimmune encephalitis (EAE) model of multiple sclerosis (see, e.g., Steinman and Zamvil (2005) TRENDS in Immunology 26(11):565-571; Ruddle et al. (1990) J. Exp. Med. 172(4):1193-1200), and a mouse model of diet-induced obesity and diabetes (see, e.g., Araujo et al. (2007) Endocrinol. 148(12):5991-5997. Examples of mouse models of human disease include mice that exhibit a disease-associated characteristic or characteristics, including, but not limited to, many forms of cancer, arthritis, multiple sclerosis, and obesity.

In certain embodiments, an animal model is a human tumor xenograft model of cancer. Human tumor xenograft models typically involve the transplantation of human tumor cells into a non-human animal, either subcutaneously or into an organ. In certain embodiments, the human tumor cells are transplanted into an immunocompromised non-human animal. In certain embodiments, the human tumor cells are transplanted into an immunocompromised mouse, and the transplanted human tumor cells typically form a primary tumor. The growth and progression (e.g., metastasis) of the primary tumor can then be monitored. In certain embodiments, the human tumor xenograft model is the JIMT-1 model of breast cancer, in which JIMT-1 human breast cancer cells are subcutaneously injected into the flank of a severe combined immunodeficient (SCID) mouse. In certain embodiments, A549, H460, Colo205, and other cell line-derived xenograft models are subcutaneously injected into the flank of a SCID mouse. In certain embodiments, the xenograft can correspond to human tumors that are directly grafted onto SCID mice (e.g., patient-derived xenograft tumor models).

### Certain Methods of Validating a Candidate Target Molecule In Vivo

The present invention provides a method of validating a candidate target molecule for a therapeutic antibody in vivo without the need for producing and purifying significant quantities of antibodies prior to validation. A variety of approaches can be used to identify a candidate target molecule. For example, a candidate target molecule can be identified based on known or predicted properties of the candidate target molecule, which suggest that it may contribute to disease. A candidate target molecule can also be identified based on experimentation, if experimental data suggest that the candidate target molecule may contribute to disease. Whether the candidate target molecule is selected based on its known, predicted, or experimentally determined properties, modulation of its activity via binding of a therapeutic antibody would be predicted to alleviate, inhibit progression, or decrease the severity of a disease.

A candidate target molecule for a therapeutic antibody may include, but is not limited to, a secreted ligand that binds to the extracellular domain (ECD) of a transmembrane receptor. Ligand binding to the ECD of the transmembrane receptor can stimulate downstream signaling events that promote disease. When a therapeutic antibody binds the ligand and inhibits ligand binding to the ECD, downstream signaling events and disease can also be inhibited. Alternatively, the ECD itself may serve as the candidate target molecule. In certain embodiments, if ligand binding to the ECD stimulates downstream signaling events that promote disease initiation and/or progression, a therapeutic antibody that binds the ECD and blocks ligand binding may inhibit disease. In certain other embodiments, if ligand binding to the ECD stimulates downstream signaling events that inhibit disease initiation and/or progression, a therapeutic antibody that binds the ECD of the transmembrane receptor and stimulates its activity may inhibit disease. In certain embodiments, if ligand binding to the ECD activates downstream signaling events by inducing receptor dimerization, therapeutic antibody binding to the ECD may prevent receptor dimerization and inhibit downstream signaling events. In certain such embodiments, inhibition of downstream signaling events could occur regardless of whether binding of the therapeutic antibody to the ECD also inhibits ligand binding to the ECD. In certain embodiments, a therapeutic antibody is an immunotoxin in which the therapeutic antibody is fused to a toxin that inhibits the growth of and/or kills the target cells. In certain embodiments, a therapeutic antibody may be interalized and bind to an intracellular target molecule, thereby inhibiting the function of the intracellular target molecule.

After a candidate target molecule is selected, an animal model of the associated disease is identified. Animal models include previously established animal models that have been published, as well as newly established animal models that have not yet been published. Typically, an animal model is identified in which modulation of candidate target molecule activity is predicted to alleviate, inhibit progression, or decrease the severity of the disease in the animal model. In certain embodiments, an animal model may exhibit increased levels and/or activity of the candidate target molecule, which would contribute to disease. In this case, inhibition of candidate target molecule levels and/or activity via therapeutic antibody binding would be expected to inhibit disease. In certain embodiments, an animal model may exhibit decreased levels and/or activity of the candidate target molecule, which would in turn contribute to disease. In this case, stimulation of candidate target molecule levels and/or activity via therapeutic antibody binding would be expected to inhibit disease. In certain embodiments, the levels and/or activity of a candidate target molecule may be unchanged in the animal model. In this case, a candidate target molecule may indirectly contribute to disease by regulating the activity of a downstream effector molecule whose activity and/or levels are either increased or decreased, thereby contributing to disease. Thus, a therapeutic antibody to the candidate target molecule would be expected to inhibit disease by blocking or reducing the candidate target molecule's effect on the downstream effector molecule.

Once a candidate target molecule and an animal model of the associated disease have been identified, antibodies to the candidate target molecule are developed using standard techniques. Nucleic acids encoding the antibody light chain and the antibody heavy chain are used. In certain examples, two separate nucleic acids, the first encoding the antibody heavy chain and the second encoding the antibody light chain, are used. In other examples, a single nucleic acid that encodes both the antibody heavy chain and the antibody light chain is used. A variety of approaches can be employed to obtain nucleic acids encoding the antibody heavy chain and the antibody light chain. In some embodiments, a hybridoma that produces a monoclonal antibody against the candidate target molecule may already exist. Alternatively, the candidate target molecule or a fragment of the candidate target molecule may be used as an antigen to generate hybridomas, which can be used to clone the corresponding nucleic acids encoding the antibody heavy chain and the antibody light chain. In certain embodiments, cells expressing the candidate target molecule on the cell surface can be used as an antigen to generate hybridomas. In certain such embodiments, mouse cells, including, but not limited to, mouse 3T3 cells, are transfected with a vector for expression of the candidate target molecule on the cell surface, and the transfected cells are used as antigens to generate hybridomas. In order to isolate hybridomas that specifically recognize the candidate target molecule, the antibodies produced by the hybridomas may be exposed in sequence to a matched set of cells, only one of which expresses the antigen of interest (e.g., a matched set of untransfected parental cells and parental cells transfected with a vector for candidate target molecule expression). In this way, hybridomas that express antibodies that bind to the untransfected parental cells can be eliminated prior to selecting for those that express antibodies that bind to the candidate target molecule expressed on the surface of the transfected cells. In certain such embodiments, antibodies that bind to the candidate target molecule but also exhibit nonspecific binding to the cell surface may be eliminated. In addition to hybridoma-based methods for generating nucleic acids encoding antibodies to a candidate target molecule, other methods, including, but not limited to, recombinant methods, genetically altered and transgenic animal-based methods, and display-based methods can be used to obtain nucleic acids encoding antibodies to a candidate target molecule.

In certain embodiments, some of the antibodies to the candidate target molecule are selected using one or more in vitro assays. As a non-limiting example, the antibodies may be assayed for their binding affinity to the candidate target molecule. In certain embodiments, only those antibodies with a particular affinity for the candidate target molecule are used in the methods described herein. Nonlimiting examples of methods for determining antibody affinity for an antigen include ELISA, immunoprecipitation, immunostaining, competitive binding assays, surface plasmon resonance, etc. In certain examples, the antibodies may be assayed for other characteristics, such as their ability to inhibit or enhance candidate target molecule function in an in vitro cell-based assay. In certain such embodiments, only those antibodies that inhibit or enhance candidate target molecule function in the in vitro cell-based assay are used in the methods described herein. One skilled in the art can select these or other assays for choosing the antibodies to be used in the methods described herein.

Once the nucleic acids encoding the antibody heavy chain and the antibody light chain are obtained, they may be cloned into a selected vector, including, but not limited to, a minicircle DNA vector, an adenoviral vector, and adeno-associated virus vector, and a lentiviral vector. In some embodiments, a single vector encodes both the antibody heavy chain and the antibody light chain. In certain other embodiments, two separate vectors are used, wherein the first vector encodes the antibody heavy chain and the second vector encodes the antibody light chain.

In order to determine the in vivo efficacy of an antibody to the candidate target molecule in an animal model, the nucleic acid(s) encoding the antibody heavy chain and the antibody light chain are delivered to the animal model using standard techniques. Nonlimiting exemplary techniques for delivering the nucleic acids include, but are not limited to, hydrodynamic delivery by tail vein injection, or by local delivery of the nucleic acid into an organ or tissue, including, but not limited to, by injection of the nucleic acid into skeletal muscle or into a tumor xenograft (see. e.g., Darquet et al. (1999) Gene Ther. 6:209-218). Hydrodynamic delivery by tail vein injection is described, e.g., in Zhang et al. (1999) Hum. Gene Ther. 10(10):1735-1737; Liu et al. (1999) Gene Therapy 6:1258-1266; Zhang et al. (2000) Gene Therapy 7:1344-1349; and U.S. Patent Application No. 2005/0153451 A1, and involves the injection of one or more nucleic acids in a large aqueous volume into the tail vein of an animal such as, for example, a mouse.

In some cases, initial experiments will be carried out to determine whether the nucleic acid(s) encoding the antibody are expressed in vivo in an animal prior to their use in an animal model. In certain embodiments, the animal is a mouse. In certain embodiments, the mouse is a SCID mouse, or other mouse lacking immunoglobulin expression. Standard assays, including, but not limited to, ELISAs and immunoblotting, can be used to detect antibody expression in plasma samples to determine whether the antibody heavy chain and the antibody light chain are expressed in vivo. The composition of the in vivo expressed antibodies can then be determined by comparing non-reduced (i.e., assembled antibodies) and reduced (e.g., treated with dithiothreitol (DTT)) plasma samples or antibodies isolated from plasma samples that are separated by non-reducing polyacrylamide gel electrophoresis. The molecular weights of the protein bands that contain antibody heavy chains and/or antibody light chains can then be determined by immunoblotting. The results of this type of experiment can be used to determine whether the in vivo expressed antibody has the expected composition. For example, if a full-length antibody was expressed in the animal, one can determine whether it is comprised of two heavy chains and two light chains, as would be expected. In certain embodiments, the ability of the in vivo expressed antibody to bind the candidate target molecule may also be determined using standard techniques. Such techniques include, but are not limited to, ELISA, immunoprecipitation experiments, and fluorescence activated cell sorting (FACS) experiments, and may be carried out, e.g., using plasma samples from the injected animal.

In certain embodiments, once it is determined that the antibody to the candidate target molecule is properly expressed in vivo and that the in vivo expressed antibody binds the candidate target molecule, experiments are carried out to determine whether the in vivo expressed antibody is efficacious in the selected animal model. In certain embodiments, the nucleic acids encoding the antibody to the candidate target molecule may be tested for in vivo efficacy before determining whether the candidate target molecule is properly expressed in vivo and whether the in vivo expressed antibody binds the candidate target molecule. Regardless of whether in vivo expression or binding is known, if the delivery of a nucleic acid(s) encoding the antibody to the candidate target molecule is efficacious in the selected animal model, the candidate target molecule is validated as being a viable target for a therapeutic antibody to treat the selected disease. This validation occurs before the time and expense are invested to produce and purify antibodies in large quantities.

In certain embodiments, an animal model is injected with two nucleic acids, wherein the first nucleic acid encodes the heavy chain and the second nucleic acid encodes the light chain of an antibody that binds the candidate target molecule. In certain other embodiments, the animal model is injected with a single nucleic acid that encodes both the heavy chain and the light chain of the antibody that binds to the candidate target molecule. The disease-associated characteristics of the animal model injected with the nucleic acid(s) encoding the antibody to the candidate target molecule are compared to those of control animals. A candidate target molecule is validated when delivery of the nucleic acid encoding the antibody that binds to the candidate target molecule leads to a decrease in disease-associated characteristics relative to control animals. In certain embodiments, the control animal is injected with a control antibody that does not bind to the candidate target molecule. In certain such embodiments, the control animal is injected with two nucleic acids, wherein the first nucleic acid encodes the heavy chain and the second nucleic acid encodes the light chain of an antibody that does not bind to the candidate target molecule. In certain embodiments, the control animal is injected with a single nucleic acid encoding both the heavy chain and the light chain, wherein the antibody does not bind to the candidate target molecule. Typically, the control antibody is expressed from a similar nucleic acid molecule as the antibody that binds to the candidate target molecule. Thus, as a nonlimiting example, if the antibody that binds to the candidate target molecule is expressed from a single minicircle DNA vector, then the control antibody is also expressed from a single minicircle DNA vector.

In certain embodiments, the invention provides methods of validating a tumor therapy target in vivo. In certain such embodiments, an animal model is selected that is a model for a human cancer. Such models include, but are not limited to, human tumor xenograft models, angiogenesis models, syngeneic mouse tumor models, and patient-derived xenograft tumor models. See, e.g., Prewett et al. (1999) Cancer Res. 59:5209-5218; WO 2009/026303, and Fiebig et al. (2007) Can. Gen. and Prot. 4:197-210. In certain embodiments, the human tumor xenograft model is the JIMT-1 model of human breast cancer. In certain embodiments, the human tumor xenograft model is the A549, H460, Colo205, or other human cell line-derived xenograft models. In certain embodiments, the candidate tumor therapy target molecule is validated when injection of the nucleic acid(s) encoding the antibody that binds the candidate target molecule leads to the inhibition of human tumor xenograft growth. In certain embodiments, inhibition of tumor xenograft growth may be determined by comparing the size of the human tumor xenograft in the animal model injected with the nucleic acid encoding the antibody to the candidate target molecule to the size of the human tumor xenograft in the animal model injected with the control antibody.

### Certain Methods of Screening Antibodies to a Selected Target Molecule In Vivo

In certain embodiments, the present invention provides methods of screening antibodies to a selected target molecule for efficaciousness in vivo without the need for producing and purifying significant quantities of antibodies prior to screening. This method can be used to screen for new antibodies to a validated target molecule, including, but not limited to, new antibodies to a validated target molecule for which a therapeutic antibody is already commercially available. This method can also be used to screen for antibodies to a candidate target molecule. This method additionally includes screening for the best optimized antibody to a target molecule after an original antibody is humanized, subjected to affinity maturation, and/or modified using other mutagenic methods. Initial screening steps may utilize Fab fragments to identify those with optimal properties, followed by the screening of full-length antibodies derived from the Fab fragments. Often, the target molecule has already been validated before the antibodies are screened. In some embodiments, a target molecule may have already been validated in humans using a different method. However, in some cases, the target molecule has not already been validated before the antibodies are screened, and thus, validation of the target molecule and screening of the antibodies to select the antibody that exhibits an appropriate balance between efficaciousness and low side effects in vivo occur during the same experiment.

Nucleic acids encoding antibodies to the selected target molecule are prepared as described above, using standard techniques, including, but not limited to, hybridoma-based methods, recombinant methods, genetically altered and transgenic mouse-based methods, and display-based methods. The nucleic acids encoding the antibody heavy chain and the antibody light chain are then cloned into an appropriate vector or vectors, as discussed above. Prior to screening an entire collection of antibodies for efficaciousness in vivo, in certain embodiments, the antibodies used for in vivo screening are selected on the basis of one or more in vitro assays. Such assays include, but are not limited to, assays to determine binding affinity, assays to determine the ability of the antibody to modulate the activity of the target molecule, assays to determine antibody efficaciousness in an in vitro assay of the disease, and assays to determine whether the antibody has other characteristics that would be expected to inhibit disease. Such in vitro assays may include, but are not limited to, assays to detect inhibition of tumor cell growth, assays to detect an increase in apoptosis, assays to monitor angiogenesis, assays to detect migration, assays to detect inhibition of signal transduction, and assays to detect the inhibition of a protein-protein interaction. Many exemplary such assays are known in the art, and an appropriate assay can be selected by one skilled in the art. See, e.g., Prewett et al. (1999) Cancer Res. 59:5209-5218; and WO 2009/026303.

The antibodies to be screened may be derived from one or more antibodies to a target molecule that have already been identified as efficacious in vivo. The antibodies to be screened may also be humanized variants of one or more antibodies to a target molecule that have already been identified as efficacious in vivo. The antibodies to be screened may also be the result of affinity maturation of one or more antibodies to a target molecule that have already been identified as efficacious in vivo. Further, the antibodies to be screened may be the result of one or more other mutagenic processes carried out on one or more antibodies to a target molecule that have already been identified as efficacious in vivo. Additionally, the antibodies to be screened may be generated by antibody chain shuffling. In some cases, the antibodies to be screened can be the result of more than one of the above processes. Additionally, the antibodies to be screened can be derived from one or more antibodies to a target molecule that have already been identified as efficacious in humans.

The nucleic acid(s) encoding the antibodies to the target molecule may be delivered to the animal model using standard techniques, including, but not limited to, hydrodynamic delivery by tail vein injection, or by local delivery of the nucleic acid into an organ or tissue, including, but not limited to, by injection of the nucleic acid into skeletal muscle or into a tumor xenograft, as described above. In certain embodiments, the expression and/or composition of the antibodies in plasma samples from the injected animals may be determined, as discussed above. The ability of the in vivo expressed antibodies to bind the target molecule may also be determined, as discussed above.

The present invention provides a method for in vivo screening of multiple antibodies to a target molecule to determine which antibody or antibodies are most efficacious in a selected animal model of disease. In certain embodiments, multiple antibodies are screened in parallel, and the results of the experiments for two or more of the antibodies are compared in order to select the antibody or antibodies that are most efficacious in vivo. For each antibody, the selected animal model is injected with a nucleic acid(s) encoding the antibody, and the ability of the in vivo expressed antibody to inhibit certain disease-associated characteristics is determined. The results of the in vivo efficacy assays for two or more of the antibodies are then compared to select the antibody or antibodies having the most desirable properties. Using this method, an antibody having particularly desirable properties in vivo can be selected without having to invest the time and expense into producing and purifying many different antibodies in significant quantities.

In certain embodiments, the present invention provides methods of screening for antibodies that exhibit lower toxicity and/or lower side effects. In certain embodiments, the present invention also provides screening methods for the early detection of toxicity and/or side effects associated with a candidate antibody. In certain such embodiments, a lead antibody may not be the most efficacious antibody, but may exhibit lower toxicity and/or decreased side effects. In certain such embodiments, an antibody with lower efficacy may be selected because it exhibits other desirable properties, including, but not limited to, lower toxicity and/or lower side effects.

In certain embodiments, the invention provides methods of screening for antibodies in which the non-antigen binding regions of the antibodies are modified in order to improve efficacy, lower toxicity, and/or decrease side effects. For example, an IgG isotype subclass may be substituted (e.g., replacing IgG1 with IgG2), or a mutated Fc may be used instead of a wild-type Fc.

In certain embodiments, the invention provides methods of screening for antibodies to a selected tumor therapy target molecule in vivo. In certain such embodiments, an animal model is selected that is a model for a human cancer. Such models include, but are not limited to, human tumor xenograft models, syngeneic mouse tumor models, and patient-derived xenograft tumor models. In certain embodiments, the human tumor xenograft model is the JIMT-1 model of human breast cancer. In certain embodiments, the human tumor xenograft model is an A549 xenograft model, an H460 xenograft model, a Colo205 xenograft model, or another human cell line-derived xenograft model. The nucleic acids encoding antibodies to be screened are prepared, as described above, and injected into the human tumor xenograft model. After a certain period of time, the injected animals are compared to each other to determine the size of the human tumor xenografts, and those antibodies that are most effective at inhibiting human tumor xenograft growth are selected. In certain embodiments, a control animal comprising the same human tumor xenograft is injected with a nucleic acid(s) encoding a control antibody that does not bind to the tumor therapy target molecule. In certain embodiments, the growth of the human tumor xenografts from the control animals is compared to the growth of the human tumor xenografts from the animal models injected with nucleic acids encoding antibodies that bind to the tumor therapy target molecule.

### EXAMPLES

The examples discussed below are intended to be purely exemplary of the invention and should not be considered to limit the invention in any way. The examples are not intended to represent that the experiments below are all or the only experiments performed.

### Example 1

### Construction of Certain Minicircle DNA Vectors for Anti-HER2 Antibody Expression

A minicircle DNA vector for use in hydrodynamic tail vein transfection (TVT) experiments that provides co-expression of the anti-HER2 antibody heavy chain and the anti-HER2 antibody light chain was constructed as follows. Clones containing the CMV promoter upstream of the anti-HER2 antibody heavy chain cDNA or the anti-HER2 antibody light chain cDNA were used as templates for PCR and traditional cloning. The amino acid sequences of the anti-HER2 antibody heavy and light chains without a signal peptide are available from Drug Bank (http://www.drugbank.ca/drugs/DB00072), and correspond to SEQ ID NO: 2 and SEQ ID NO: 5, respectively. The amino acid sequences of the signal peptides for the anti-HER2 antibody heavy chain and light chain, which are derived from a mouse antibody locus, are identical and correspond to SEQ ID NO: 3 and SEQ ID NO: 6, respectively. The nucleic acid sequences of the signal peptides for the anti-HER2 antibody heavy chain and light chain are also identical and correspond to SEQ ID NO: 9 and SEQ ID NO: 12, respectively.

The nucleic acid sequence encoding the anti-HER2 antibody heavy chain includes a signal peptide, a variable region, a constant region, and a human IgG1 Fc, and is shown in SEQ ID NO: 10. That nucleic acid sequence encodes the anti-HER2 antibody heavy chain amino acid sequence shown in SEQ ID NO: 4.

The nucleic acid sequence encoding the anti-HER2 antibody light chain includes a signal peptide, a variable region, a constant region, and a human IgG1 Fc, and is shown in SEQ ID NO: 13. That nucleic acid sequence encodes the anti-HER2 antibody light chain amino acid sequence shown in SEQ ID NO: 7.

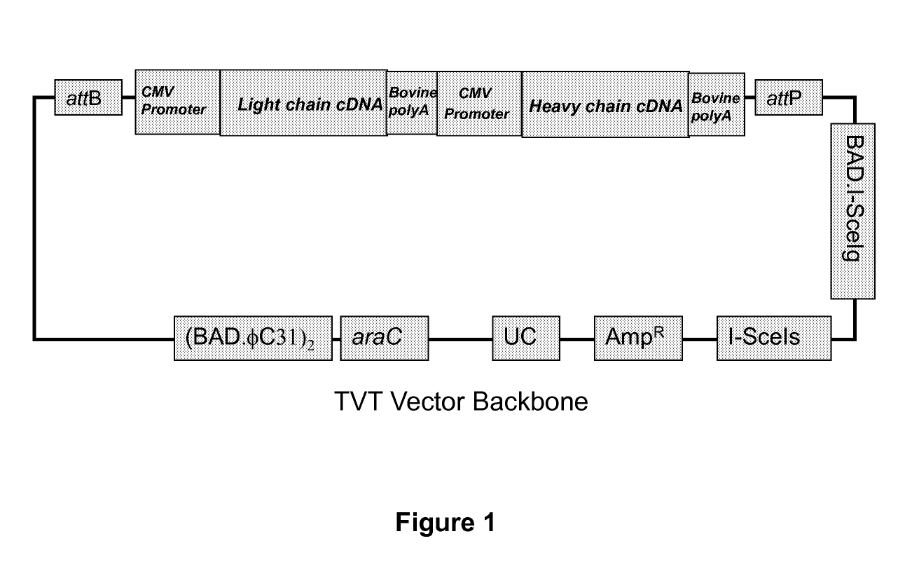

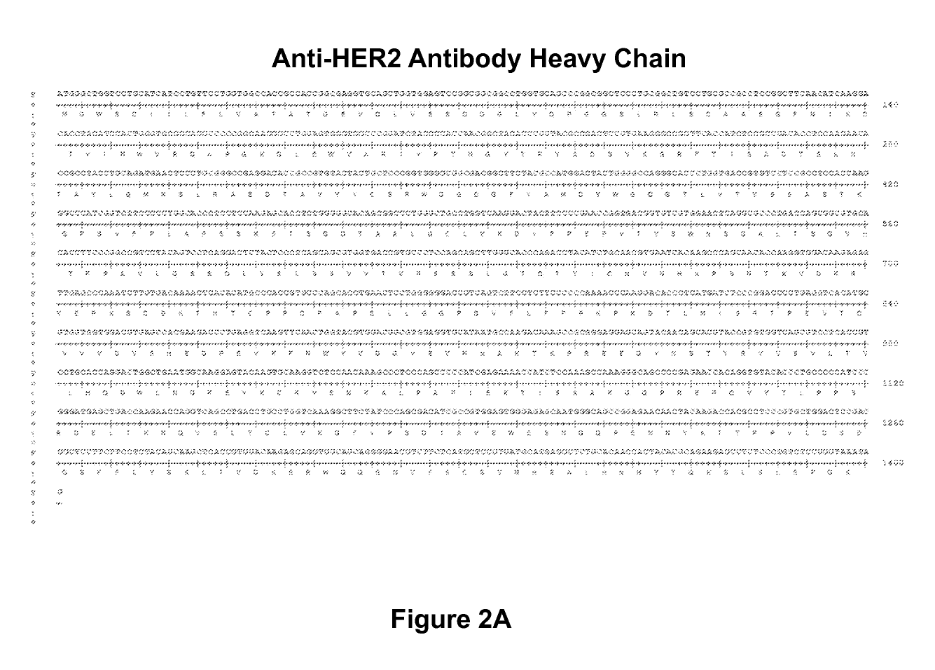

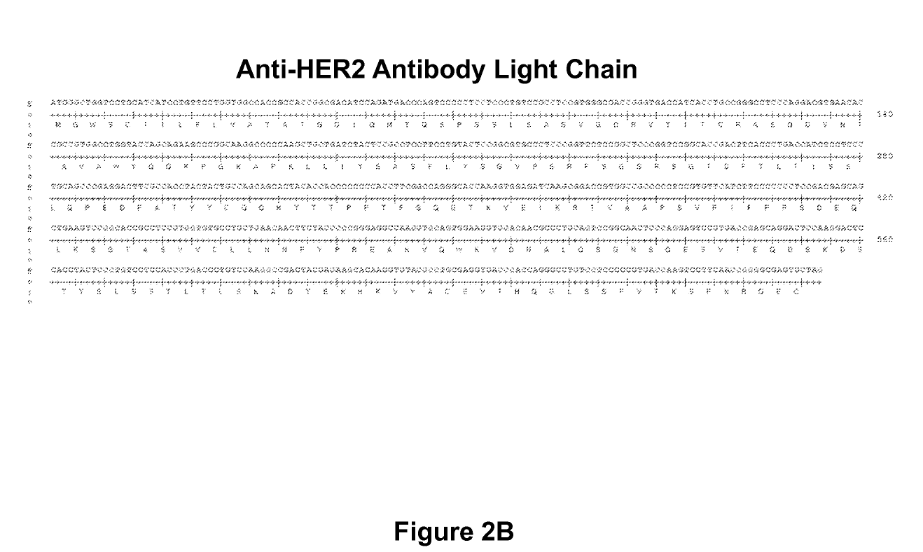

The nucleic acid sequence of the CMV promoter, including the beta-globin intron, is derived from the pCMV-MCS vector (Stratagene, La Jolla, Calif.), and is shown in SEQ ID NO: 1. Nucleic acids encoding the anti-HER2 antibody heavy chain and light chain, each with an upstream CMV promoter and beta-globin intron, were inserted into a single minicircle DNA vector, as shown in FIG. 1. The minicircle DNA vector used in these experiments is based on the p2φC31.hFIX minicircle DNA vector of Chen et al. (2005) Human Gene Therapy 16(1):126-131. Specifically, the minicircle DNA vector is comprised of the φC31 integrase gene (φC31), the bacterial attachment site (attB), the phage attachment site (attP), the BAD promoter (BAD), the araC repressor (araC), the ampicillin resistance gene (Amp^{R}), the pUC plasmid replication origin (UC), the I-SceI gene (I-SceIg), and the I-SceI cutting site (I-SceIs). FIG. 1 shows a diagram of a minicircle DNA vector for expression of anti-HER2 antibody. The vector includes nucleic acids encoding the antibody heavy chain and the antibody light chain, each preceded by the CMV promoter and beta-globin intron and followed by a bovine poly(A) tail. The nucleic acid sequence of the bovine poly(A) tail is shown in SEQ ID NO: 14. FIGS. 2A and 2B show the nucleic acid and amino acid sequences for the anti-HER2 antibody heavy chain and light chain, respectively.

### Example 2

### In Vivo Expression and Composition of Anti-HER2 Antibody by Hydrodynamic Tail Vein Transfection

Experiments were carried out to determine whether hydrodynamic tail vein injection of the minicircle DNA vector encoding anti-HER2 antibody, shown in FIG. 1, would lead to anti-HER2 antibody expression in vivo in mice. The minicircle DNA vector of FIG. 1 is prepared as follows. A single bacterial colony is grown in 5 ml Luria-Bertani (LB) broth containing 100 ug/ml carbenicillin (LB/Carb) for 6-8 hours at 37° C., 250 rpm, followed by overnight growth and amplification in 500 ml LB/Carb at 37° C., 250 rpm. The following day, the bacteria are spun down at 1,600×g for 20 min at 20° C., resuspended in 125 ml LB (pH 7.0) containing 1% L-arabinose, and incubated for two hours at 32° C., 250 rpm. Following the incubation, 62.5 ml of low salt 0.05% LB (pH 8.0) containing 1% arabinose was added to the bacterial culture, and the culture was incubated for two hours at 37° C., 250 rpm. The bacteria were centrifuged at 6,000×g for 15 min. The minicircle DNA vector was then isolated using the Qiagen Plasmid Mega Kit (Qiagen Inc., Valencia, Calif.).

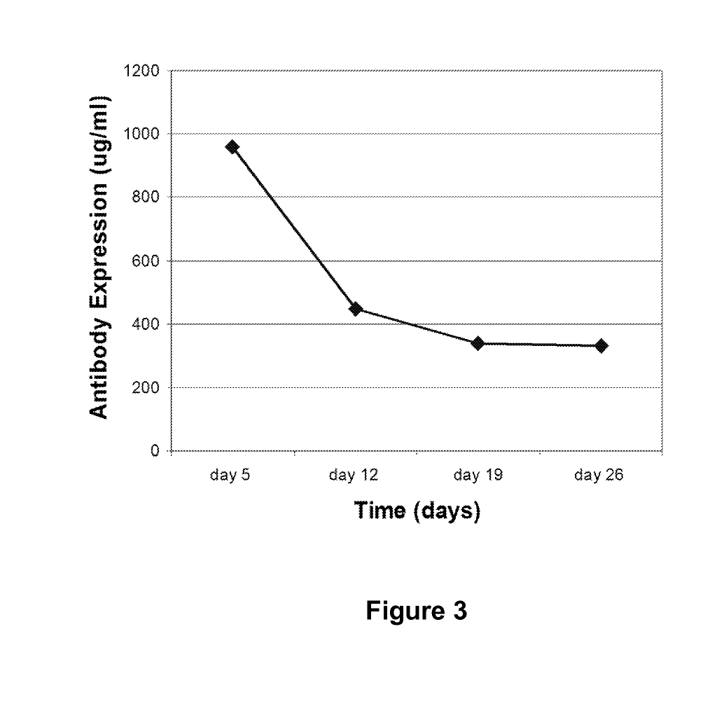

Three severe combined immunodeficient (SCID) mice (6-8 weeks of age) were hydrodynamically transfected by tail vain injection with the minicircle DNA vector encoding anti-HER2 antibody (20 ug/ml in 2 ml saline containing 0.91% w/v NaCl). Plasma samples were collected on days 5, 12, 19, and 26 post-injection. An ELISA assay to detect human Fc was performed to determine anti-HER2 antibody levels in the plasma samples. Briefly, ELISA plates were coated with 50 ul/well of 4 ng/ml polyclonal goat anti-human IgG-Fc affinity purified capture antibody (Bethyl Labs # A80-104A), followed by an overnight incubation at 4° C. Plasma samples were diluted 1:80, 1:400, 1:2,000, and 1:10,000 in dilution buffer (PBS containing 1% BSA). The capture antibody solutions were discarded, and 150 ul/well of blocking solution (PBS containing 1% BSA) was added, followed by shaking for one hour at room temperature. After blocking, the diluted plasma samples, positive control samples (purified anti-HER2 antibody), and negative control samples (PBS containing 0.05% Tween-20, “PBST”) were added to the wells. The plates were then incubated for two hours at room temperature without shaking, followed by three washes with PBST. Fifty microliters of polyclonal goat anti-human IgG-Fc HRP-conjugated probe antibody (Bethyl Labs #A80-104P, stock 1 mg/ml diluted 1:60,000 in PBS) were added to each well, and the plates were incubated at room temperature for one hour without shaking. SuperSignal ELISA Pico Chemiluminescent Substrate (Pierce #37069) was used to detect the signal. FIG. 3 shows the results of the ELISA experiments to determine anti-HER2 antibody expression levels in plasma from SCID mice following hydrodynamic tail vein injection with the minicircle DNA vector shown in FIG. 1. This experiment showed that injection with the minicircle DNA vector encoding anti-HER2 antibody led to high, sustained anti-HER2 antibody expression in vivo.

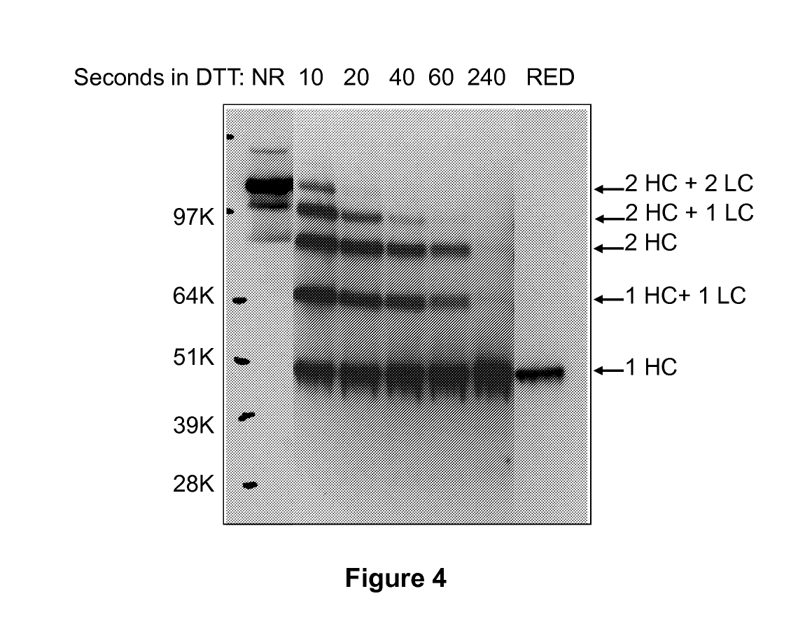

Experiments were carried out to determine the composition of the anti-HER2 antibody expressed in vivo. Mouse plasma samples (0.03 ul) were diluted into 15 ul 1×SDS sample buffer (BioRad #161-0791) containing 10 mM DTT, subjected to reducing conditions at room temperature for selected time periods (10, 20, 40, 60, and 240 sec), and the reactions were quenched by the addition of 15 ul 1×SDS sample buffer containing 40 mM iodoacetamide. Samples were heated to 70° C. for 10 min, separated by non-reducing polyacrylamide gel electrophoresis, and subjected to immunoblot analysis using a polyclonal goat anti-human IgG-Fc HRP conjugated antibody (Bethyl Labs #A80-104P). The fully reduced sample was quenched after the 70° C. treatment. FIG. 4 shows an immunoblot from the time-course experiment with DTT treatment used to determine anti-HER2 antibody composition in plasma from a SCID mouse hydrodynamically transfected by tail vein injection with the minicircle DNA vector encoding anti-HER2 antibody. The results of this experiment show that the anti-HER2 antibody expressed in vivo formed a complex comprised of two heavy chains and two light chains, as expected.

### Example 3

### Binding of Anti-HER2 Antibody from TVT Plasma Samples to the HER2 Cell Surface Antigen of HT-B-30 Cells

Experiments were carried out to determine the binding activity of anti-HER2 antibody in two plasma samples from SCID mice injected with the minicircle DNA vector encoding anti-HER2 antibody on the surface of HT-B-30 (ATCC Number HTB-30) cells. HT-B-30 cells, which express the HER2 antigen on their cell surface, were cultured in McCoy 5A medium (Mediatech Inc, Manassas, Va.; Cat. No. 10-050-CV) containing 10% FBS (Mediatech Inc, Manassas, Va.; Cat. No. 35-0,1-CV). The HT-B-30 cells were washed twice with PBS (without Ca²⁺ or Mg²⁺), and were incubated in 3 mM EDTA (37° C., 2-3 min) to detach the cells. The cells were centrifuged (1,000 rpm, 5 min, in a Thermo Electric Co., CentraCL2 centrifuge) and were resuspended at a density of approximately 5×10⁶ cells/ml in Buffer A (PBS containing 1% BSA and 0.1% NaN₃).

HT-B-30 cells were incubated with IgG1 (1 mg/ml, 100 ug/ml, 10 ug/ml, 1 ug/ml, and 0.1 ug/ml in Buffer A) as a negative control, with purified anti-HER2 antibody (1 mg/ml, 100 ug/ml, 10 ug/ml, 1 ug/ml, and 0.1 ug/ml in Buffer A) as a positive control, with negative control plasma (undiluted, or diluted 1:10, 1:100, or 1:1,000 in Buffer A), or with plasma samples from two SCID mice hydrodynamically transfected by tail vein injection with the minicircle DNA vector encoding anti-HER2 antibody (undiluted, or diluted 1:10, 1:100, or 1:1,000 in Buffer A). Twenty microliters of each of the above diluted protein or plasma samples were added to 200 ul aliquots of the HT-B-30 cells that each contained 1×10⁶ cells. This resulted in a series of dilutions of the plasma samples (1:10, 1:100, 1:1,000, and 1:10,000) and protein solutions (100 ug/ml, 10 ug/ml, 1 ug/ml, 0.1 ug/ml, and 0.01 ug/ml), each of which was incubated with 1×10⁶ HT-B-30 cells.

The cells were incubated with the diluted plasma or protein samples for 30 min at 4° C., followed by centrifugation at 1,200 rpm for 5 min (Thermo Electric Co., CentraCL2 centrifuge) and three washes with PBS. In order to detect cell surface-bound anti-HER2 antibody, the cells were resuspended in 200 ul of Buffer B (PBS containing 1% BSA and 10 ug/ml FITC-labeled anti-IgG antibody (Jackson ImmunoResearch Laboratories, Inc., West Grove, Pa., Cat No. 109-096-170)). Following a 30 min incubation at 4° C., the cells were washed three times with PBS. After the final centrifugation step, the cells were resuspended in 200 ul PBS containing propidium iodide (PI) (1:500 dilution, BD Pharmingen, San Diego, Calif.; Cat #556463). FACS analysis was performed to determine the level of cell surface HER2 antigen binding by anti-HER2 antibody in the plasma samples. FACS data from the purified anti-HER2 antibody positive control were used to generate a standard curve, which was used to determine the HER2 binding activity of the two plasma samples.

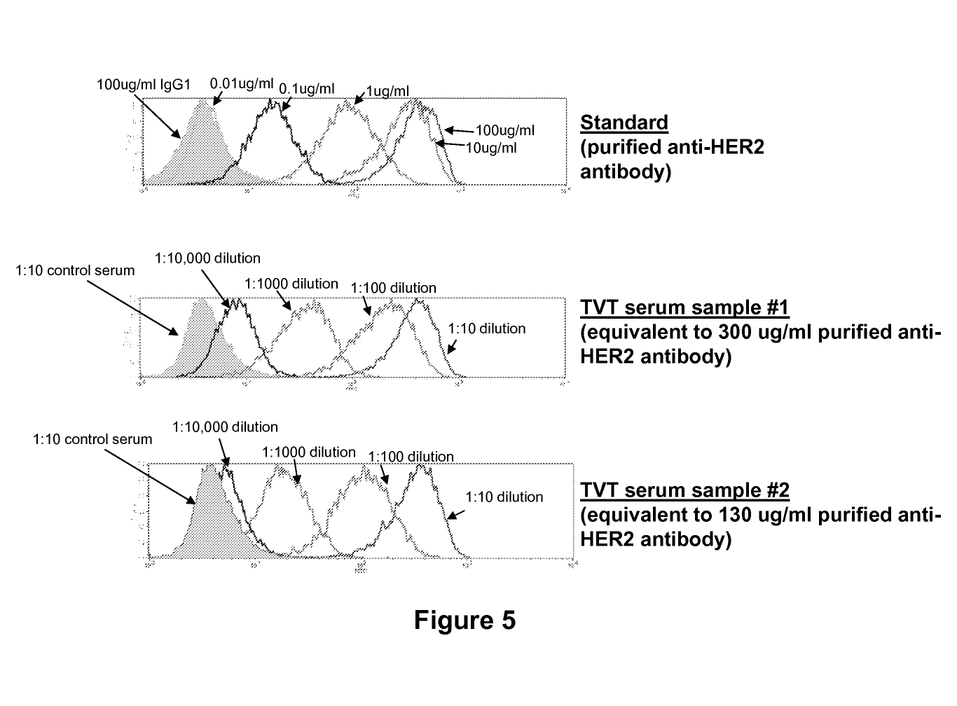

The two plasma samples from mice injected with the minicircle DNA vector encoding anti-HER2 exhibited HER2 binding activity equivalent to that of 300 ug/ml and 130 ug/ml of the purified anti-HER2 antibody positive control. FIG. 5 shows the results of the FACS analysis to determine the HER2 binding activity of anti-HER2 antibody in the two plasma samples from SCID mice hydrodynamically transfected by tail vein injection with the minicircle DNA vector shown in FIG. 1 to HER2 on the surface of HT-B-30 cells. The results of this experiment demonstrated that anti-HER2 antibody in the plasma samples bound to HER2 on the surface of HT-B-30 cells.

### Example 4

### Hydrodynamic Tail Vein Injection of a Minicircle DNA Vector Encoding Anti-HER2 Antibody Inhibits Tumor Growth in the JIMT-1 Xenograft Model of Breast Cancer

Experiments were carried out to determine whether hydrodynamic tail vein injection of the minicircle DNA vector encoding anti-HER2 antibody could inhibit primary tumor growth in the JIMT-1 xenograft model of breast cancer. The JIMT-1 model. See e.g., Barok et al., (2007) Mol. Cancer Ther. 6(7):2065-2072. The minicircle DNA vector encoding anti-HER2 antibody, shown in FIG. 1, was used in these experiments. The minicircle DNA vector encoding anti-HER2 antibody was purified and injected as described in Example 2.

Female SCID mice (Charles River Laboratories) (6-8 weeks of age) were weighed, ear tagged, and divided randomly into one of four treatment groups, shown in Table 1 below:

TABLE 1
Treatment groups for JIMT-1 xenograft mouse model experiments.

| Group | n | Route | Treatment | Dose | | Schedule |
|---|---|---|---|---|---|---|
| 1 | 10 | i.p. | Albumin | 1 | mg/kg | 3x/wk × |
| | | | (negative control | | | 4 wks |
| | | | group) | | | |
| 2 | 10 | i.p. | Purified Anti-HER2 | 1 | mg/kg | 3x/wk × |
| | | | Antibody (positive | | | 4 wks |
| | | | control group) | | | |
| 3 | 12 | TVT | Saline (negative | 2 | ml | once |
| | | | control group) | | | |

| | | | | | |
|---|---|---|---|---|---|
| 4 | 12 | TVT | Minicircle DNA | 20 ug DNA/ | once |
| | | | Encoding Anti-HER2 | mouse in 2 | |
| | | | Antibody (experi- | ml saline | |
| | | | mental group) | | |

JIMT-1 cells (Deutsche Sammlung von Mikroorganismen and Zellkulturen GmbH; DSMZ No.: ACC 589) were cultured in DMEM medium containing 10% FBS and 1% L-glutamine at 37° C. in a humidified atmosphere with 5% CO₂. When the cultured cells were between 85-90% confluent, they were harvested and resuspended in cold PBS (without Ca²⁺ or Mg²⁺) containing 50% Matrigel at a density of 5×10⁷ cells/ml. The cells were implanted subcutaneously over the right flank at 5×10⁶ cells/100 ul/mouse. Dosing for treatment groups 1 and 2 began one day after tumor implantation and involved intraperitoneal (i.p.) injection with 1 mg/kg control albumin and 1 mg/kg purified anti-HER2 antibody, respectively, three times a week for four weeks. Dosing for treatment groups 3 and 4 was performed five days before tumor implantation and involved a single hydrodynamic tail vein injection with 2 ml saline and 2 ml saline containing 20 ug minicircle DNA encoding anti-HER2 antibody, respectively. The length (L) and width (W) of each tumor were measured using an electronic caliper, and the volume (V) of each tumor was calculated using the formula V=(L×W²)/2. Tumor volume and body weight were measured weekly.

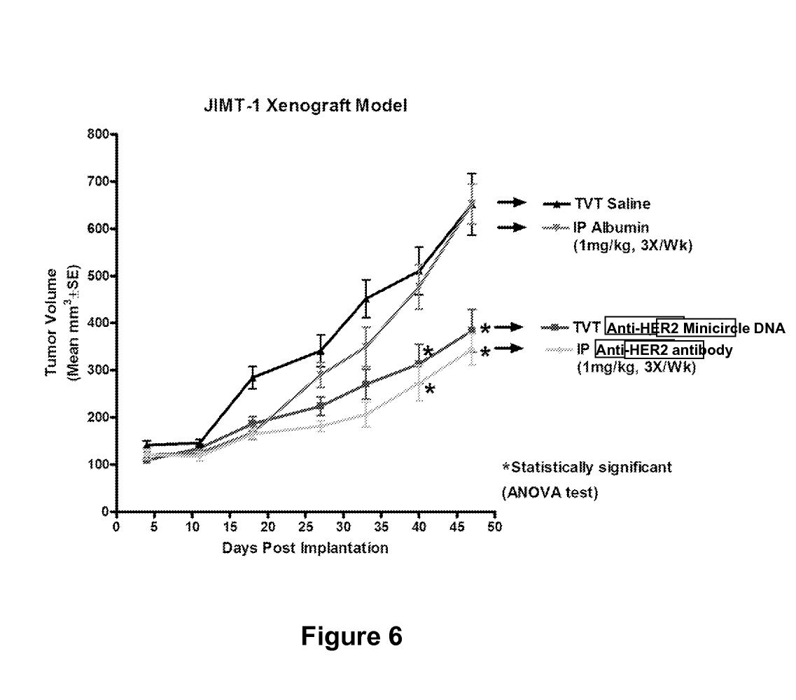

FIG. 6 shows the results of experiments to determine whether hydrodynamic tail vein injection of the minicircle DNA vector encoding anti-HER2 antibody inhibits primary tumor growth in the JIMT-1 xenograft model of breast cancer. The ANOVA test indicates statistical significance (*, #). These experiments showed that hydrodynamic tail vein injection of the minicircle DNA vector encoding anti-HER2 antibody inhibited primary tumor growth in the JIMT-1 xenograft model of breast cancer.

### TABLE OF SEQUENCES

Table 2 provides certain sequences discussed herein.

TABLE 2
Sequences and Descriptions

| SEQ. | | |
|---|---|---|
| ID. NO. | Description | Sequence |
| 1 | CMV Promoter With the | CTAGTTATTA ATAGTAATCA ATTACGGGGT |
| | Beta-Globin Intron | CATTAGTTCA TAGCCCATAT ATGGAGTTCC |
| | Nucleic Acid Sequence | GCGTTACATA ACTTACGGTA AATGGCCCGC |
| | from pCMV-MCS | CTGGCTGACC GCCCAACGAC CCCCGCCCAT |
| | (Stratagene) | TGACGTCAAT AATGACGTAT GTTCCCATAG |
| | | TAACGTCAAT AGGGACTTTC CATTGACGTC |
| | | AATGGGTGGA GTATTTACGG TAAACTGCCC |
| | | ACTTGGCAGT ACATCAAGTG TATCATATGC |
| | | CAAGTACGCC CCCTATTGAC GTCAATGACG |
| | | GTAAATGGCC CGCCTGGCAT TATGCCCAGT |
| | | ACATGACCTT ATGGGACTTT CCTACTTGGC |
| | | AGTACATCTA CGTATTAGTC ATCGCTATTA |
| | | CCATGGTGAT GCGGTTTTGG CAGTACATCA |
| | | ATGGGCGTGG ATAGCGGTTT GACTCACGGG |
| | | GATTTCCAAG TCTCCACCCC ATTGACGTCA |
| | | ATGGGAGTTT GTTTTGCACC AAAATCAACG |
| | | GGACTTTCCA AAATGTCGTA ACAACTCCGC |
| | | CCCATTGACG CAAATGGGCG GTAGGCGTGT |
| | | ACGGTGGGAG GTCTATATAA GCAGAGCTCG |
| | | TTTAGTGAAC CGTCAGATCG CCTGGAGACG |
| | | CCATCCACGC TGTTTTGACC TCCATAGAAG |
| | | ACACCGGGAC CGATCCAGCC TCCGCGGATT |
| | | CGAATCCCGG CCGGGAACGG TGCATTGGAA |
| | | CGCGGATTCC CCGTGCCAAG AGTGACGTAA |
| | | GTACCGCCTA TAGAGTCTAT AGGCCCACAA |
| | | AAAATGCTTT CTTCTTTTAA TATACTTTTT |
| | | TGTTTATCTT ATTTCTAATA CTTTCCCTAA |
| | | TCTCTTTCTT TCAGGGCAAT AATGATACAA |
| | | TGTATCATGC CTCTTTGCAC CATTCTAAAG |
| | | AATAACAGTG ATAATTTCTG GGTTAAGGCA |
| | | ATAGCAATAT TTCTGCATAT AAATATTTCT |
| | | GCATATAAAT TGTAACTGAT GTAAGAGGTT |
| | | TCATATTGCT AATAGCAGCT ACAATCCAGC |
| | | TACCATTCTG CTTTTATTTT ATGGTTGGGA |
| | | TAAGGCTGGA TTATTCTGAG TCCAAGCTAG |
| | | GCCCTTTTGC TAATCATGTT CATACCTCTT |
| | | ATCTTCCTCC CACAGCTCCT GGGCAACGTG |
| | | CTGGTCTGTG TGCTGGCCCA TCACTTTGGC |
| | | AAAGAATTGG GATTCGAACA TCGATTGAAT |
| | | TCCCCAAACT TAAGCTT |
| 2 | Anti-HER2 Antibody | EVQLVESGGG LVQPGGSLRL SCAASGFNIK |
| | Heavy Chain Protein | DTYIHWVRQA PGKGLEWVAR IYPTNGYTRY |
| | Sequence Without a | ADSVKGRFTI SADTSKNTAY LQMNSLRAED |
| | Signal Peptide, derived | TAVYYCSRWG GDGFYAMDYW GQGTLVTVSS |
| | from DrugBank | ASTKGPSVFP LAPSSKSTSG GTAALGCLVK |
| | | DYFPEPVTVS WNSGALTSGV HTFPAVLQSS |
| | | GLYSLSSVVT VPSSSLGTQT YICNVNHKPS |
| | | NTKVDKKVEP PKSCDKTHTC PPCPAPELLG |
| | | GPSVFLFPPK PKDTLMISRT PEVTCVVVDV |
| | | SHEDPEVKFN WYVDGVEVHN AKTKPREEQY |
| | | NSTYRVVSVL TVLHQDWLNG KEYKCKVSNK |
| | | ALPAPIEKTI SKAKGQPREP QVYTLPPSRD |
| | | ELTKNQVSLT CLVKGFYPSD IAVEWESNGQ |
| | | PENNYKTTPP VLDSDGSFFL YSKLTVDKSR |
| | | WQQGNVFSCS VMHEALHNHY TQKSLSLSPG |
| | | K |
| 3 | Anti-HER2 Antibody | MGWSCIILFL VATATG |
| | Heavy Chain Signal | |
| | Peptide Protein Sequence | |
| 4 | Anti-HER2 Antibody | MGWSCIILFL VATATGEVQL VESGGGLVQP |
| | Heavy Chain Protein | GGSLRLSCAA SGFNIKDTYI HWVRQAPGKG |
| | Sequence With a Signal | LEWVARIYPT NGYTRYADSV KGRFTISADT |
| | Peptide | SKNTAYLQMN SLRAEDTAVY YCSRWGGDGF |
| | | YAMDYWGQGT LVTVSSASTK GPSVFPLAPS |
| | | SKSTSGGTAA LGCLVKDYFP EPVTVSWNSG |
| | | ALTSGVHTFP AVLQSSGLYS LSSVVTVPSS |
| | | SLGTQTYICN VNHKPSNTKV DKKVEPPKSC |
| | | DKTHTCPPCP APELLGGPSV FLFPPKPKDT |
| | | LMISRTPEVT CVVVDVSHED PEVKFNWYVD |
| | | GVEVHNAKTK PREEQYNSTY RVVSVLTVLH |
| | | QDWLNGKEYK CKVSNKALPA PIEKTISKAK |
| | | GQPREPQVYT LPPSRDELTK NQVSLTCLVK |
| | | GFYPSDIAVE WESNGQPENN YKTTPPVLDS |
| | | DGSFFLYSKL TVDKSRWQQG NVFSCSVMHE |
| | | ALHNHYTQKS LSLSPGK |
| 5 | Anti-HER2 Antibody | DIQMTQSPSS LSASVGDRVT ITCRASQDVN |
| | Light Chain Protein | TAVAWYQQKP GKAPKLLIYS ASFLYSGVPS |
| | Sequence Without a | RFSGSRSGTD FTLTISSLQP EDFATYYCQQ |
| | Signal Peptide, derived | HYTTPPTFGQ GTKVEIKRTV AAPSVFIFPP |
| | from DrugBank | SDEQLKSGTA SVVCLLNNFY PREAKVQWKV |
| | | DNALQSGNSQ ESVTEQDSKD STYSLSSTLT |
| | | LSKADYEKHK VYACEVTHQG LSSPVTKSFN |
| | | RGEC |
| 6 | Anti-HER2 Antibody | MGWSCIILFL VATATG |
| | Light Chain Signal | |
| | Peptide Protein Sequence | |
| 7 | Anti-HER2 Antibody | MGWSCIILFL VATATGDIQM TQSPSSLSAS |
| | Light Chain Protein | VGDRVTITCR ASQDVNTAVA WYQQKPGKAP |
| | Sequence With a Signal | KLLIYSASFL YSGVPSRFSG SRSGTDFTLT |
| | Peptide | ISSLQPEDFA TYYCQQHYTT PPTFGQGTKV |
| | | EIKRTVAAPS VFIFPPSDEQ LKSGTASVVC |
| | | LLNNFYPREA KVQWKVDNAL QSGNSQESVT |
| | | EQDSKDSTYS LSSTLTLSKA DYEKHKVYAC |
| | | EVTHQGLSSP VTKSFNRGEC |
| 8 | Anti-HER2 Antibody | GAGGTGCAGC TGGTGGAGTC CGGCGGCGGC |
| | Heavy Chain Nucleic | CTGGTGCAGC CCGGCGGCTC CCTGCGGCTG |
| | Acid Sequence Without a | TCCTGCGCCG CCTCCGGCTT CAACATCAAG |
| | Signal Peptide, derived | GACACCTACA TCCACTGGGT GCGGCAGGCC |
| | from DrugBank | CCCGGCAAGG GCCTGGAGTG GGTGGCCCGG |
| | | ATCTACCCCA CCAACGGCTA CACCCGGTAC |
| | | GCCGACTCCG TGAAGGGCCG GTTCACCATC |
| | | TCCGCCGACA CCTCCAAGAA CACCGCCTAC |
| | | CTGCAGATGA ACTCCCTGCG GGCCGAGGAC |
| | | ACCGCCGTGT ACTACTGCTC CCGGTGGGGC |
| | | GGCGACGGCT TCTACGCCAT GGACTACTGG |
| | | GGCCAGGGCA CCCTGGTGAC CGTGTCCTCC |
| | | GCCTCCACCA AGGGCCCATC GGTCTTCCCC |
| | | CTGGCACCCT CCTCCAAGAG CACCTCTGGG |
| | | GGCACAGCGG CCCTGGGCTG CCTGGTCAAG |
| | | GACTACTTCC CCGAACCGGT GACGGTGTCG |
| | | TGGAACTCAG GCGCCCTGAC CAGCGGCGTG |
| | | CACACCTTCC CGGCCGTCCT ACAGTCCTCA |
| | | GGACTCTACT CCCTCAGCAG CGTGGTGACC |
| | | GTGCCCTCCA GCAGCTTGGG CACCCAGACC |
| | | TACATCTGCA ACGTGAATCA CAAGCCCAGC |
| | | AACACCAAGG TGGACAAGAG AGTTGAGCCC |
| | | AAATCTTGTG ACAAAACTCA CACATGCCCA |
| | | CCGTGCCCAG CACCTGAACT CCTGGGGGGA |
| | | CCGTCAGTCT TCCTCTTCCC CCCAAAACCC |
| | | AAGGACACCC TCATGATCTC CCGGACCCCT |
| | | GAGGTCACAT GCGTGGTGGT GGACGTGAGC |
| | | CACGAAGACC CTGAGGTCAA GTTCAACTGG |
| | | TACGTGGACG GCGTGGAGGT GCATAATGCC |
| | | AAGACAAAGC CGCGGGAGGA GCAGTACAAC |
| | | AGCACGTACC GTGTGGTCAG CGTCCTCACC |
| | | GTCCTGCACC AGGACTGGCT GAATGGCAAG |
| | | GAGTACAAGT GCAAGGTCTC CAACAAAGCC |
| | | CTCCCAGCCC CCATCGAGAA AACCATCTCC |
| | | AAAGCCAAAG GGCAGCCCCG AGAACCACAG |
| | | GTGTACACCC TGCCCCCATC CCGGGATGAG |
| | | CTGACCAAGA ACCAGGTCAG CCTGACCTGC |
| | | CTGGTCAAAG GCTTCTATCC CAGCGACATC |
| | | GCCGTGGAGT GGGAGAGCAA TGGGCAGCCG |
| | | GAGAACAACT ACAAGACCAC GCCTCCCGTG |
| | | CTGGACTCCG ACGGCTCCTT CTTCCTCTAC |
| | | AGCAAGCTCA CCGTGGACAA GAGCAGGTGG |
| | | CAGCAGGGGA ACGTCTTCTC ATGCTCCGTG |
| | | ATGCATGAGG CTCTGCACAA CCACTACACG |
| | | CAGAAGAGCC TCTCCCTGTC TCCGGGTAAA |
| | | TAG |
| 9 | Anti-HER2 Antibody | ATGGGCTGGT CCTGCATCAT CCTGTTCCTG |
| | Heavy Chain Signal | GTGGCCACCG CCACCGGC |
| | Peptide Nucleic Acid | |
| | Sequence | |
| 10 | Anti-HER2 Antibody | ATGGGCTGGT CCTGCATCAT CCTGTTCCTG |
| | Heavy Chain Nucleic | GTGGCCACCG CCACCGGCGA GGTGCAGCTG |
| | Acid Sequence With a | GTGGAGTCCG GCGGCGGCCT GGTGCAGCCC |
| | Signal Peptide | GGCGGCTCCC TGCGGCTGTC CTGCGCCGCC |
| | | TCCGGCTTCA ACATCAAGGA CACCTACATC |
| | | CACTGGGTGC GGCAGGCCCC CGGCAAGGGC |
| | | CTGGAGTGGG TGGCCCGGAT CTACCCCACC |
| | | AACGGCTACA CCCGGTACGC CGACTCCGTG |
| | | AAGGGCCGGT TCACCATCTC CGCCGACACC |
| | | TCCAAGAACA CCGCCTACCT GCAGATGAAC |
| | | TCCCTGCGGG CCGAGGACAC CGCCGTGTAC |
| | | TACTGCTCCC GGTGGGGCGG CGACGGCTTC |
| | | TACGCCATGG ACTACTGGGG CCAGGGCACC |
| | | CTGGTGACCG TGTCCTCCGC CTCCACCAAG |
| | | GGCCCATCGG TCTTCCCCCT GGCACCCTCC |
| | | TCCAAGAGCA CCTCTGGGGG CACAGCGGCC |
| | | CTGGGCTGCC TGGTCAAGGA CTACTTCCCC |
| | | GAACCGGTGA CGGTGTCGTG GAACTCAGGC |
| | | GCCCTGACCA GCGGCGTGCA CACCTTCCCG |
| | | GCCGTCCTAC AGTCCTCAGG ACTCTACTCC |
| | | CTCAGCAGCG TGGTGACCGT GCCCTCCAGC |
| | | AGCTTGGGCA CCCAGACCTA CATCTGCAAC |
| | | GTGAATCACA AGCCCAGCAA CACCAAGGTG |
| | | GACAAGAGAG TTGAGCCCAA ATCTTGTGAC |
| | | AAAACTCACA CATGCCCACC GTGCCCAGCA |
| | | CCTGAACTCC TGGGGGGACC GTCAGTCTTC |
| | | CTCTTCCCCC CAAAACCCAA GGACACCCTC |
| | | ATGATCTCCC GGACCCCTGA GGTCACATGC |
| | | GTGGTGGTGG ACGTGAGCCA CGAAGACCCT |
| | | GAGGTCAAGT TCAACTGGTA CGTGGACGGC |
| | | GTGGAGGTGC ATAATGCCAA GACAAAGCCG |
| | | CGGGAGGAGC AGTACAACAG CACGTACCGT |
| | | GTGGTCAGCG TCCTCACCGT CCTGCACCAG |
| | | GACTGGCTGA ATGGCAAGGA GTACAAGTGC |
| | | AAGGTCTCCA ACAAAGCCCT CCCAGCCCCC |
| | | ATCGAGAAAA CCATCTCCAA AGCCAAAGGG |
| | | CAGCCCCGAG AACCACAGGT GTACACCCTG |
| | | CCCCCATCCC GGGATGAGCT GACCAAGAAC |
| | | CAGGTCAGCC TGACCTGCCT GGTCAAAGGC |
| | | TTCTATCCCA GCGACATCGC CGTGGAGTGG |
| | | GAGAGCAATG GGCAGCCGGA GAACAACTAC |
| | | AAGACCACGC CTCCCGTGCT GGACTCCGAC |
| | | GGCTCCTTCT TCCTCTACAG CAAGCTCACC |
| | | GTGGACAAGA GCAGGTGGCA GCAGGGGAAC |
| | | GTCTTCTCAT GCTCCGTGAT GCATGAGGCT |
| | | CTGCACAACC ACTACACGCA GAAGAGCCTC |
| | | TCCCTGTCTC CGGGTAAA |
| 11 | Anti-HER2 Antibody | GACATCCAGA TGACCCAGTC CCCCTCCTCC |
| | Light Chain Nucleic Acid | CTGTCCGCCT CCGTGGGCGA CCGGGTGACC |
| | Sequence Without a | ATCACCTGCC GGGCCTCCCA GGACGTGAAC |
| | Signal Peptide, derived | ACCGCCGTGG CCTGGTACCA GCAGAAGCCC |
| | from DrugBank | GGCAAGGCCC CCAAGCTGCT GATCTACTCC |
| | | GCCTCCTTCC TGTACTCCGG CGTGCCCTCC |
| | | CGGTTCTCCG GCTCCCGGTC CGGCACCGAC |
| | | TTCACCCTGA CCATCTCCTC CCTGCAGCCC |
| | | GAGGACTTCG CCACCTACTA CTGCCAGCAG |
| | | CACTACACCA CCCCCCCCAC CTTCGGCCAG |
| | | GGCACCAAGG TGGAGATCAA GCGGACCGTG |
| | | GCCGCCCCCT CCGTGTTCAT CTTCCCCCCC |
| | | TCCGACGAGC AGCTGAAGTC CGGCACCGCC |
| | | TCCGTGGTGT GCCTGCTGAA CAACTTCTAC |
| | | CCCCGGGAGG CCAAGGTGCA GTGGAAGGTG |
| | | GACAACGCCC TGCAGTCCGG CAACTCCCAG |
| | | GAGTCCGTGA CCGAGCAGGA CTCCAAGGAC |
| | | TCCACCTACT CCCTGTCCTC CACCCTGACC |
| | | CTGTCCAAGG CCGACTACGA GAAGCACAAG |
| | | GTGTACGCCT GCGAGGTGAC CCACCAGGGC |
| | | CTGTCCTCCC CCGTGACCAA GTCCTTCAAC |
| | | CGGGGCGAGT GC |
| 12 | Anti-HER2 Antibody | ATGGGCTGGT CCTGCATCAT CCTGTTCCTG |
| | Light Chain Signal | GTGGCCACCG CCACCGGC |
| | Peptide Nucleic Acid | |
| | Sequence | |
| 13 | Anti-HER2 Antibody | ATGGGCTGGT CCTGCATCAT CCTGTTCCTG |
| | Light Chain Nucleic Acid | GTGGCCACCG CCACCGGCGA CATCCAGATG |
| | Sequence With a Signal | ACCCAGTCCC CCTCCTCCCT GTCCGCCTCC |
| | Peptide | GTGGGCGACC GGGTGACCAT CACCTGCCGG |
| | | GCCTCCCAGG ACGTGAACAC CGCCGTGGCC |
| | | TGGTACCAGC AGAAGCCCGG CAAGGCCCCC |
| | | AAGCTGCTGA TCTACTCCGC CTCCTTCCTG |
| | | TACTCCGGCG TGCCCTCCCG GTTCTCCGGC |
| | | TCCCGGTCCG GCACCGACTT CACCCTGACC |
| | | ATCTCCTCCC TGCAGCCCGA GGACTTCGCC |
| | | ACCTACTACT GCCAGCAGCA CTACACCACC |
| | | CCCCCCACCT TCGGCCAGGG CACCAAGGTG |
| | | GAGATCAAGC GGACCGTGGC CGCCCCCTCC |
| | | GTGTTCATCT TCCCCCCCTC CGACGAGCAG |
| | | CTGAAGTCCG GCACCGCCTC CGTGGTGTGC |
| | | CTGCTGAACA ACTTCTACCC CCGGGAGGCC |
| | | AAGGTGCAGT GGAAGGTGGA CAACGCCCTG |
| | | CAGTCCGGCA ACTCCCAGGA GTCCGTGACC |
| | | GAGCAGGACT CCAAGGACTC CACCTACTCC |
| | | CTGTCCTCCA CCCTGACCCT GTCCAAGGCC |
| | | GACTACGAGA AGCACAAGGT GTACGCCTGC |
| | | GAGGTGACCC ACCAGGGCCT GTCCTCCCCC |
| | | GTGACCAAGT CCTTCAACCG GGGCGAGTGC |
| 14 | Bovine Poly(A) Tail | GCTCGCTGAT CAGCCTCGAC TGTGCCTTCT |
| | Nucleic Acid Sequence | AGTTGCCAGC CATCTGTTGT TTGCCCCTCC |
| | | CCCGTGCCTT CCTTGACCCT GGAAGGTGCC |
| | | ACTCCCACTG TCCTTTCCTA ATAAAATGAG |
| | | GAAATTGCAT CGCATTGTCT GAGTAGGTGT |
| | | CATTCTATTC TGGGGGGTGG GGTGGGGCAG |
| | | GACAGCAAGG GGGAGGATTG GGAAGACAAT |
| | | AGCAGGCATG CTGGGGATGC GGTGGGCTCT |
| | | ATGGCTTCTG AGGCGGAAAG AANCCAGCTG |
| | | GGGCTCGAGG GCCATACAGG CCGGTACC |

1-28. (canceled)

29. A method of screening a plurality of therapeutic antibodies to a target molecule in vivo, comprising:
a) obtaining a plurality of compositions, wherein each composition comprises a heavy chain nucleic acid that encodes an antibody heavy chain and a light chain nucleic acid that encodes an antibody light chain, wherein the antibody binds to the target molecule;
b) injecting a first composition into a first mouse comprising a first human tumor xenograft, wherein the first composition comprises:
i) a first heavy chain nucleic acid that encodes a first antibody heavy chain; and
ii) a first light chain nucleic acid that encodes a first antibody light chain; and
iii) wherein the first antibody binds to the target molecule;
c) injecting a second composition into a second mouse comprising a second human tumor xenograft, wherein the second composition comprises:
i) a second heavy chain nucleic acid that encodes a second antibody heavy chain; and
ii) a second light chain nucleic acid that encodes a second antibody light chain; and
iii) wherein the second antibody binds to the target molecule;
d) measuring the sizes of the first and second human tumor xenografts after a period of time; and
e) comparing the sizes of the first and second human tumor xenografts; and
f) selecting the antibody that results in the smaller human tumor xenograft.

30. The method of claim 29, wherein the first heavy chain nucleic acid encodes a first antibody heavy chain variable region, and the first light chain nucleic acid encodes a first antibody light chain variable region, and wherein the second heavy chain nucleic acid encodes a second antibody heavy chain variable region, and the second light chain nucleic acid encodes a second antibody light chain variable region.

31. The method of claim 29, wherein the first heavy chain nucleic acid encodes a first antibody heavy chain variable region and a first antibody heavy chain constant region, and the first light chain nucleic acid encodes a first antibody light chain variable region and a first antibody light chain constant region, and wherein the second heavy chain nucleic acid encodes a second antibody heavy chain variable region and a second antibody heavy chain constant region, and the second light chain nucleic acid encodes a second antibody light chain variable region and a second antibody light chain constant region.

32. The method of claim 29, wherein the injecting comprises hydrodynamic transfection.

33. The method of claim 32, wherein the injecting comprises hydrodynamic tail vein transfection.

34. The method of claim 29, wherein the first heavy chain nucleic acid is a first minicircle DNA vector, and the first light chain nucleic acid is a second minicircle DNA vector, and wherein the second heavy chain nucleic acid is a third minicircle DNA vector, and the second light chain nucleic acid is a fourth minicircle DNA vector.

35. A method of screening a plurality of therapeutic antibodies to a target molecule in vivo, comprising:
a) obtaining a plurality of compositions, wherein each composition comprises a nucleic acid that encodes an antibody heavy chain and an antibody light chain, wherein the antibody binds to the target molecule;
b) injecting a first composition into a first mouse comprising a first human tumor xenograft, wherein the first composition comprises a first nucleic acid that encodes a first antibody heavy chain and a first antibody light chain, wherein the first antibody binds to the target molecule;
c) injecting a second composition into a second mouse comprising a second human tumor xenograft, wherein the second composition comprises a second nucleic acid that encodes a second antibody heavy chain and a second antibody light chain, wherein the second antibody binds to the target molecule;
d) measuring the sizes of the first and second human tumor xenografts after a period of time; and
e) comparing the sizes of the first and second human tumor xenografts; and
f) selecting the antibody that results in the smaller human tumor xenograft.

36. The method of claim 35, wherein the first nucleic acid encodes a first antibody heavy chain variable region and a first antibody light chain variable region, and wherein the second nucleic acid encodes a second antibody heavy chain variable region and a second antibody light chain variable region.

37. The method of claim 36, wherein the first nucleic acid encodes a first antibody heavy chain variable region, a first antibody light chain variable region, and a first flexible linker that connects the first antibody heavy chain variable region and the first antibody light chain variable region, and wherein the second nucleic acid encodes a second antibody heavy chain variable region, a second antibody light chain variable region, and a second flexible linker that connects the second antibody heavy chain variable region and the second antibody light chain variable region.

38. The method of claim 35, wherein the first nucleic acid encodes a first antibody heavy chain variable region and a first antibody heavy chain constant region; and a first antibody light chain variable region and a first antibody light chain constant region, and wherein the second nucleic acid encodes a second antibody heavy chain variable region and a second antibody heavy chain constant region; and a second antibody light chain variable region and a second antibody light chain constant region.

39. The method of claim 35, wherein the injecting comprises hydrodynamic transfection.

40. The method of claim 39, wherein the injecting comprises hydrodynamic tail vein transfection.

41. The method of claim 35, wherein the first nucleic acid is a first minicircle DNA vector, and wherein the second nucleic acid is a second minicircle DNA vector.

42. A method of validating a candidate target molecule in vivo, comprising:
a) injecting a composition into a mouse with a disease, wherein the composition comprises:
i) a first nucleic acid that encodes an antibody heavy chain; and
ii) a second nucleic acid that encodes an antibody light chain; and
iii) wherein the antibody binds to the candidate target molecule;
b) determining the alleviation, inhibition of progression, or decrease in severity of the disease after a period of time; and
c) comparing the alleviation, inhibition of progression, or decrease in severity of the disease to a control mouse; and
d) validating the candidate target molecule if the alleviation, inhibition of progression, or decrease in severity of the disease is greater than the alleviation, inhibition of progression, or decrease in severity of the disease in the control mouse.

43. A method of validating a candidate target molecule in vivo, comprising:
a) injecting a composition into a mouse with a disease, wherein the composition comprises a nucleic acid that encodes an antibody heavy chain and an antibody light chain, wherein the antibody binds to the candidate target molecule;
b) determining the alleviation, inhibition of progression, or decrease in severity of the disease after a period of time; and
c) comparing the alleviation, inhibition of progression, or decrease in severity of the disease to a control mouse; and
d) validating the candidate target molecule if the alleviation, inhibition of progression, or decrease in severity of the disease is greater than the alleviation, inhibition of progression, or decrease in severity of the disease in the control mouse.

44. A method of screening a plurality of therapeutic antibodies to a target molecule in vivo, comprising:
a) obtaining a plurality of compositions, wherein each composition comprises a heavy chain nucleic acid that encodes an antibody heavy chain and a light chain nucleic acid that encodes an antibody light chain, wherein the antibody binds to the target molecule;
b) injecting a first composition into a first mouse with a disease, wherein the first composition comprises:
i) a first heavy chain nucleic acid that encodes a first antibody heavy chain; and
ii) a first light chain nucleic acid that encodes a first antibody light chain; and
iii) wherein the first antibody binds to the target molecule;
c) injecting a second composition into a second mouse with the disease, wherein the second composition comprises:
i) a second heavy chain nucleic acid that encodes a second antibody heavy chain; and
ii) a second light chain nucleic acid that encodes a second antibody light chain; and
iii) wherein the second antibody binds to the target molecule;
d) determining the alleviation, inhibition of progression, or decrease in severity of the disease after a period of time; and
e) comparing the alleviation, inhibition of progression, or decrease in severity of the disease of the first and second mice; and
f) selecting the antibody that results in the greater alleviation, inhibition of progression, or decrease in severity of the disease.

45. A method of screening a plurality of therapeutic antibodies to a target molecule in vivo, comprising:
a) obtaining a plurality of compositions, wherein each composition comprises a nucleic acid that encodes an antibody heavy chain and an antibody light chain, wherein the antibody binds to the target molecule;
b) injecting a first composition into a first mouse with a disease, wherein the first composition comprises a first nucleic acid that encodes a first antibody heavy chain and a first antibody light chain, wherein the first antibody binds to the target molecule;
c) injecting a second composition into a second mouse with the disease, wherein the second composition comprises a second nucleic acid that encodes a second antibody heavy chain and a second antibody light chain, wherein the second antibody binds to the target molecule;
d) determining the alleviation, inhibition of progression, or decrease in severity of the disease after a period of time; and
e) comparing the alleviation, inhibition of progression, or decrease in severity of the disease of the first and second mice; and
f) selecting the antibody that results in the greater alleviation, inhibition of progression, or decrease in severity of the disease.
